(19) United States
(12) Patent Application Publication
(10) Pub. No.: US 2022/0098228 A1
(43) Pub. Date: Mar. 31, 2022

(54) METHODS AND COMPOSITIONS FOR IMPROVING THE IMMUNE SYSTEM AND PROVIDING IMMUNO-PROTECTION

(71) Applicant: Natreon, Inc., New Brunswick, NJ (US)

(72) Inventor: Sanyasi R Kalidindi, Monroe, NJ (US)

(73) Assignee: Natreon, Inc., New Brunswick, NJ (US)

(21) Appl. No.: 17/486,586

(22) Filed: Sep. 27, 2021

Related U.S. Application Data
(60) Provisional application No. 63/084,342, filed on Sep. 28, 2020.

(51) Int. Cl. C07H 17/04; A61P 37/04

(52) U.S. Cl. CPC C07H 17/04; A61P 37/04

(57) ABSTRACT

This invention is directed to compositions comprising dibenzo-alpha-pyrones (DBPs, Urolithins) and DBP glycosides, and methods of improving the immune system, providing immuno-protection, and/or treating immunosuppression in a subject with the compositions. The compounds 3-OH-DBP (Urolithin B), 3,8-(OH)₂-DBP (Urolithin A), and their glycosides are discussed.

### CROSS-REFERENCE TO RELATED APPLICATIONS

This application claims the benefit of U.S. Provisional Application No. 63/084,342, filed Sep. 28, 2020, which is incorporated by reference in its entirety herein.

### FIELD OF INVENTION

This invention relates to compositions comprising dibenzo-alpha-pyrones (DBPs, Urolithins) and DBP glycosides, and methods of improving the immune system, providing immuno-protection, and/or treating immunosuppression in a subject with the compositions. The compounds 3-OH-DBP (Urolithin B), 3,8-(OH)₂-DBP (Urolithin A), and their glycosides are discussed.

### BACKGROUND

The immune system helps the body fight infection and other diseases with components such as cells, tissues, organs, and various substances. An immune response is generally how immune system components react to an infectious agent or vector or the like, which is not recognized as a constituent of the body. An immune response includes responding to antigens such as those on bacteria, viruses, and other potentially harmful agents.

Immune system dysfunction is responsible for the pathophysiology of many diseases. The modulation of immune responses, either by stimulation of the immune system or by suppression of undesired immune reactions, may be helpful in alleviating such diseases. Immunomodulatory therapy could provide an alternative to conventional therapy for a variety of conditions, especially where a host's defense mechanisms have to be activated or otherwise stimulated under the conditions of impaired immune responsiveness (Singh V K, Dwivedi P, Chaudhary B R, Singh R. “Immunomodulatory Effect of Gymnema sylvestre (R. Br.) Leaf Extract: An In Vitro Study in Rat Model” PLoS One 10(10):e0139631. doi: 10.1371/journal.pone.0139631 (2015)). Cyclophosphamide may be used as a treatment to suppress the immune system, for instance following organ transplantation, and as a model for studying immunosuppression.

Shilajit is a natural substance found in mountain rocks during peak summer months. It is found at high altitudes ranging from 1000 to 5000 meters. Active constituents of Shilajit include dibenzo-alpha-pyrones (DBPs, also known as “urolithins”). 3-hydroxy-dibenzo-alpha-pyrone (3-OH-DBP, Urolithin B) and 3,8-dihydroxy-dibenzo-alpha-pyrone (3,8-(OH)₂-DBP, Urolithin A) are components of Shilajit. Shilajit also includes for instance DBP-related metabolites, small peptides (constituting non-protein amino acids), some lipids, and carrier molecules, including for instance fulvic acids, humic acids, minerals. PrimaVie® (available from Natreon, Inc., New Brunswick, N.J.) is a standardized Shilajit extract formulated as a potent and very safe dietary supplement, restoring the body's energetic balance and potentially able to prevent several diseases.

### SUMMARY OF INVENTION

The present invention is directed in part to DBP glycosides and their preparation and use. 3-OH-DBP, 3-OH-DBP glycoside, 3,8-(OH)₂-DBP, and/or 3,8-(OH)₂-DBP glycoside, and other compounds, are used according to this invention to improve the immune system, provide immuno-protection, and to treat immunosuppression. The present invention is also directed to methods for improving a subject's immune system, comprising the steps of (a) providing a composition comprising at least one of 3-OH-DBP, 3-OH-DBP glycoside, 3,8-(OH)₂-DBP, and 3,8-(OH)₂-DBP glycoside; and (b) administering the composition to the subject in an amount effective to deliver the at least one of 3-OH-DBP, 3-OH-DBP glycoside, 3,8-(OH)₂-DBP, and 3,8-(OH)₂-DBP glycoside to the subject's cells, tissues, and/or bloodstream and to act at said cells, tissues, and/or bloodstream to improve an immune system parameter.

The present invention is also directed to methods for providing immuno-protection to a subject, comprising the steps of (a) providing a composition comprising at least one of 3-OH-DBP, 3-OH-DBP glycoside, 3,8-(OH)₂-DBP, and 3,8-(OH)₂-DBP glycoside; and (b) administering the composition to the subject in an amount effective to deliver the at least one of 3-OH-DBP, 3-OH-DBP glycoside, 3,8-(OH)₂-DBP, and 3,8-(OH)₂-DBP glycoside to the subject's cells, tissues, and/or bloodstream and to act at said cells, tissues, and/or bloodstream to maintain the subject's immune response at normal healthy levels in the presence of an immunosuppressant.

The present invention is also directed to DBP glycoside compounds and compositions, and processes for preparing the DPB glycosides.

### BRIEF DESCRIPTION OF THE DRAWINGS

FIG. 1 represents an ESI-mass spectrum of tetra-O-acetyl glycopyranosyl bromide according to this invention.

FIG. 2 represents a 1H NMR spectrum of 3,8-(OH)₂-DBP glycoside of this invention.

FIG. 3 represents an ESI-mass spectrum of 3,8-(OH)₂-DBP glycoside of this invention.

FIG. 4 represents an ESI-mass spectrum of 3-OH-DBP glycoside of this invention.

### DETAILED DESCRIPTION

The below definitions and discussion are intended to guide understanding but are not intended to be limiting with regard to other disclosures in this application. References to percentage (%) in compositions of the present invention are to the % by weight of a given component to the total weight of the composition being discussed, also signified by “w/w”, unless stated otherwise.

3-OH-DBP, 3-OH-DBP glycoside, 3,8-(OH)₂-DBP, and/or 3,8-(OH)₂-DBP glycoside are used according to this invention to improve the immune system, provide immuno-protection, and to treat immunosuppression. Their structures are generally represented by Formula (I) below.

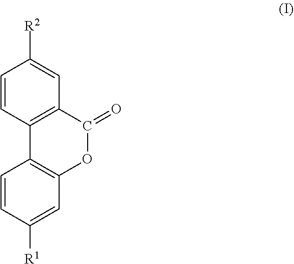

In an embodiment, R¹═OH or —O-sugar and R²═H, OH, or —O-sugar, where the sugar may be for example glucose or as otherwise described in this application. In an embodiment, Formula (I) represents 3-hydroxy-dibenzo-alpha-pyrone (3-OH-DBP, Urolithin B), where R¹═OH, R²═H.

In an embodiment, Formula (I) represents 3-O-D-Glucopyranosyl-Dibenzo-alpha-pyrone (3-OH-DBP glycoside, also called 3-OH-DBP glucoside because the sugar moiety is glucose), where R¹═—O-glucose, R²═H, shown as Formula (II) below.

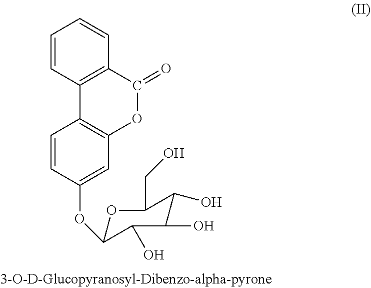 [SMILES: O=c1oc2cc(OC3OC(CO)C(O)C(O)C3O)ccc2c2ccccc12]

In an embodiment, Formula (I) represents 3,8-dihydroxy-dibenzo-alpha-pyrone (3,8-(OH)₂-DBP, Urolithin A), where R¹═OH, R²═OH.

In an embodiment, Formula (I) represents 3,8-di-(O-D-Glucopyranosyl)-Dibenzo-alpha-pyrone (3,8-(OH)₂-DBP glycoside, also called 3,8-(OH)₂-DBP glucoside because the sugar moiety is glucose), where R¹ and R²═—O-glucose, shown as Formula (III) below.

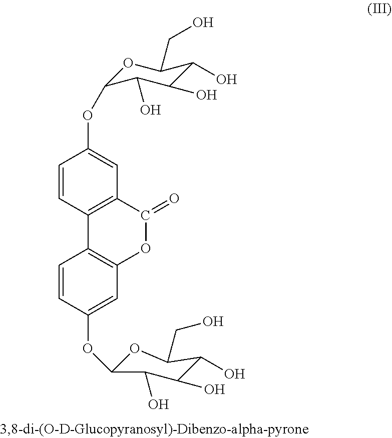 [SMILES: O=c1oc2cc(OC3OC(CO)C(O)C(O)C3O)ccc2c2ccc(OC3OC(CO)C(O)C(O)C3O)cc12]

DBP glycosides according to this invention comprise a combination of a dibenzopyrone (DBP) and a sugar such as glucose. In an embodiment, a sugar according to this invention is a monosaccharide such as glucose, xylose, rhamnose, arabinose, or galactose. In an embodiment, a sugar according to this invention is a disaccharide such as a combination of glucose, rhamnose, arabinose, xylose, and/or galactose. For instance, a disaccharide may be a combination of 2 glucose molecules, or one xylose and one galactose molecule, and so forth. In an embodiment, in a DBP glycoside according to this invention having more than one sugar, each sugar is independently a monosaccharide or a disaccharide. For instance, in a glycoside having two sugar moieties, each sugar may be a monosaccharide such as glucose, for instance alpha-D-glucose or beta-D-glucose (as shown in Formula II), or for instance one sugar may be a monosaccharide such as galactose at one point of attachment and the other sugar may be a disaccharide such as of rhamnose and xylose at another point of attachment; or each sugar may be a disaccharide. A DBP glycoside of this invention may be synthesized by adding a sugar moiety to a DBP via a glycosidic bond. The resulting DBP glycoside may be purified, identified and characterized for instance as described below. In an embodiment, a DBP glycoside of this invention is a DBP glucoside.

A “composition” of the present invention includes one or more of 3-OH-DBP, 3-OH-DBP glycoside, 3,8-(OH)₂-DBP, and/or 3,8-(OH)₂-DBP glycoside. In an embodiment, a composition of this invention comprises, consists essentially of, or consists of 3-OH-DBP, 3-OH-DBP glycoside, 3,8-(OH)₂-DBP, and/or 3,8-(OH)₂-DBP glycoside. In an embodiment, the 3-OH-DBP, 3-OH-DBP glycoside, 3,8-(OH)₂-DBP, and/or 3,8-(OH)₂-DBP glycoside are in purified form, for instance, greater than 90% pure w/w, greater than 95% pure w/w, 97-99% pure w/w, or at least 99% pure w/w. In an embodiment, the 3-OH-DBP, 3-OH-DBP glycoside, 3,8-(OH)₂-DBP, and/or 3,8-(OH)₂-DBP glycoside are synthesized or are from a natural source (if a natural source exists). In an embodiment, 3-OH-DBP of this invention is a white free-flowing powder, 3-OH-DBP glycoside is a light brown free-flowing powder, 3,8-(OH)₂-DBP is a light yellow free-flowing powder, and 3,8-(OH)₂-DBP glycoside is a brown free-flowing powder.

In an embodiment, a composition of the present invention may include or may be a synergistic composition comprising a synergistic combination of two or more of 3-OH-DBP, 3-OH-DBP glycoside, 3,8-(OH)₂-DBP, and/or 3,8-(OH)₂-DBP glycoside. In an embodiment, a synergistic combination is 3-OH-DBP and 3,8-(OH)₂-DBP combined, for instance in a ratio of 3-OH-DBP: 3,8-(OH)₂-DBP of about 1:3 to about 1:7. In an embodiment, the synergistic combination is a ratio of 3-OH-DBP: 3,8-(OH)₂-DBP of about 1:5. In an embodiment, a composition comprising 3-OH-DBP and 3,8-(OH)₂-DBP may be indicated as combined in a composition and/or for administration with a “plus” (“+”) symbol, as 3-OH-DBP+3,8-(OH)₂-DBP; others may be similarly marked. In an embodiment, without being bound by theory or indicating similar activities between DBPs and their glycosides, 3-OH-DBP glycoside may be used with or in place of 3-OH-DBP, and/or 3,8-(OH)₂-DBP glycoside may be used with or in place of 3,8-(OH)₂-DBP, or vice versa. In an embodiment, without being bound by theory or indicating dissimilar activities between DBPs and their glycosides, 3-OH-DBP glycoside may not be used with or in place of 3-OH-DBP, and/or 3,8-(OH)₂-DBP glycoside may not be used with or in place of 3,8-(OH)₂-DBP, or vice versa.

In an embodiment, a composition of this invention is PrimaVie® Shilajit (Natreon, Inc., New Brunswick, N.J.), in its original form or enriched with DBPs (including e.g. 3-OH-DBP, 3-OH-DBP glycoside, 3,8-(OH)₂-DBP, and/or 3,8-(OH)₂-DBP glycoside) for instance as disclosed throughout this application. In an embodiment, Shilajit according to this invention is a standardized aqueous extract of Shilajit (“standardized aqueous Shilajit extract”) containing at least 50% (w/w) fulvic acids with DBP core nucleus and at least 10.3% (w/w) of free DBP plus DBP conjugated with chromoproteins, and for instance more than 40 microminerals. A standardized aqueous Shilajit extract according to this invention is “PrimaVie® Shilajit”. PrimaVie® is a registered US trademark under which standardized aqueous Shilajit extract is sold. PrimaVie® Shilajit is described in U.S. Pat. Nos. 6,869,612 and 6,440,436, each of which is incorporated by reference herein for the purpose of describing PrimaVie® Shilajit, in an embodiment.

In a further embodiment, said standardized aqueous Shilajit extract (“PrimaVie® Shilajit”) is a dry powder, having the appearance of a fine, brown to dark-brown, free-flowing powder; and further conforms to one or more of the following parameters: a water-soluble extractive value of at least 80% w/w; water in an amount of 0-6% (w/w); E₄/E₆ at 465/665 nm of 7.0-9.5; pH (2.0% aqueous dispersion) at least 5; lead at no more than 2 ppm, arsenic at no more than 3 ppm, and mercury at no more than 0.5 ppm; and a microbiological profile having not more than 5000 CFU/g aerobic bacteria (USP<2021>), not more than 1000 CFU/g yeast and mold (USP<2021>), with Escherichia coli (AOAC 991.14), Pseudomonas aeruginosa (USP<62>), Staphylococcus aureus (USP<2022>), and Candida albicans (custom) all absent in 1 g of said dry powder and Salmonella species (modified AOAC 998.09) absent in 10 g of said dry powder. In an embodiment, the standardized aqueous Shilajit extract of the present invention conforms to all of these parameters.

In a further embodiment, said standardized aqueous Shilajit extract is in dry powder form and further conforms to one or more of the following parameters: a water-soluble extractive value of about 93-96% (w/w), combined DBPs of about 14-17% (w/w), Fulvic acids with DBP core nucleus of about 59-62% (w/w), water in an amount of about 1-4% (w/w), E₄/E₆ at 465/665 nm at about 7-8, pH (2.0% aqueous dispersion) at about 6-8, lead in an amount less than 1 ppm, arsenic in an amount less than 0.6 ppm, mercury in an amount less than 0.01 ppm, less than about 500 CFU/g aerobic bacteria, less than 100 CFU/g yeast and mold, with E. coli, Salmonella, Pseudomonas aeruginosa, Staphylococcus aureus, and Candida albicans not measurably present. In an embodiment, the standardized aqueous Shilajit extract of the present invention conforms to all of these parameters.

In a further embodiment, said standardized aqueous Shilajit extract is in dry powder form and conforms to one or more of the following parameters: a water-soluble extractive value of 94.5% (w/w), combined DBPs of 15.67% (w/w), fulvic acids with DBP core nucleus of 60.81% (w/w), water in an amount of 2.75% (w/w), E₄/E₆ at 465/665 nm of 7.22, pH (2.0% aqueous dispersion) of 7.41, lead in an amount of 0.882 ppm, arsenic in an amount of 0.406 ppm, mercury in an amount less than 0.004 ppm, aerobic bacteria measured at 250 CFU/g or less, and yeast and mold at less than 70 CFU/g. E. coli, Salmonella, Pseudomonas aeruginosa, Staphylococcus aureus, and Candida albicans are not measurably present. In an embodiment, the standardized aqueous Shilajit extract of the present invention conforms to all of these parameters.

HPLC (High Pressure Liquid Chromatography) and HPTLC (High Pressure Thin Layer Chromatography) may be used to confirm the conformance of a standardized aqueous Shilajit extract of the present invention to e.g. fulvic acid and DBP parameters above, and other parameters as appropriate. In an embodiment, the standardized aqueous Shilajit extract in dry powder form is stable for 3 years or more. In an embodiment, the powdered extract is stored at 15° C. to 25° C., and in a container that avoids exposure of the powdered extract to light. Other laboratory techniques may be used to characterize DBPs of this invention, for instance as shown in the Figures.

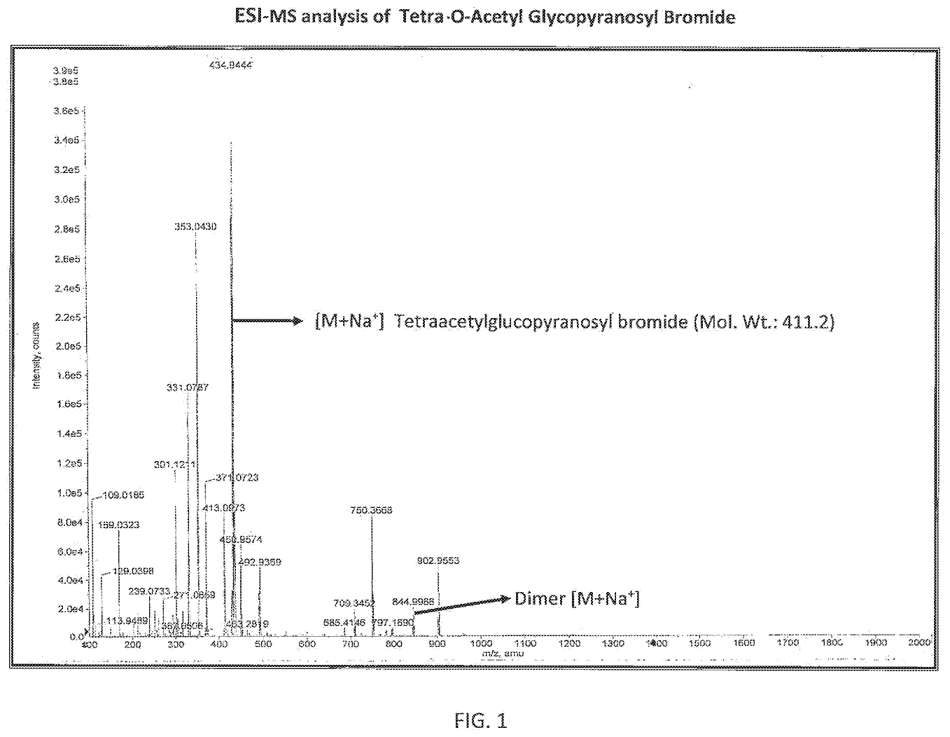

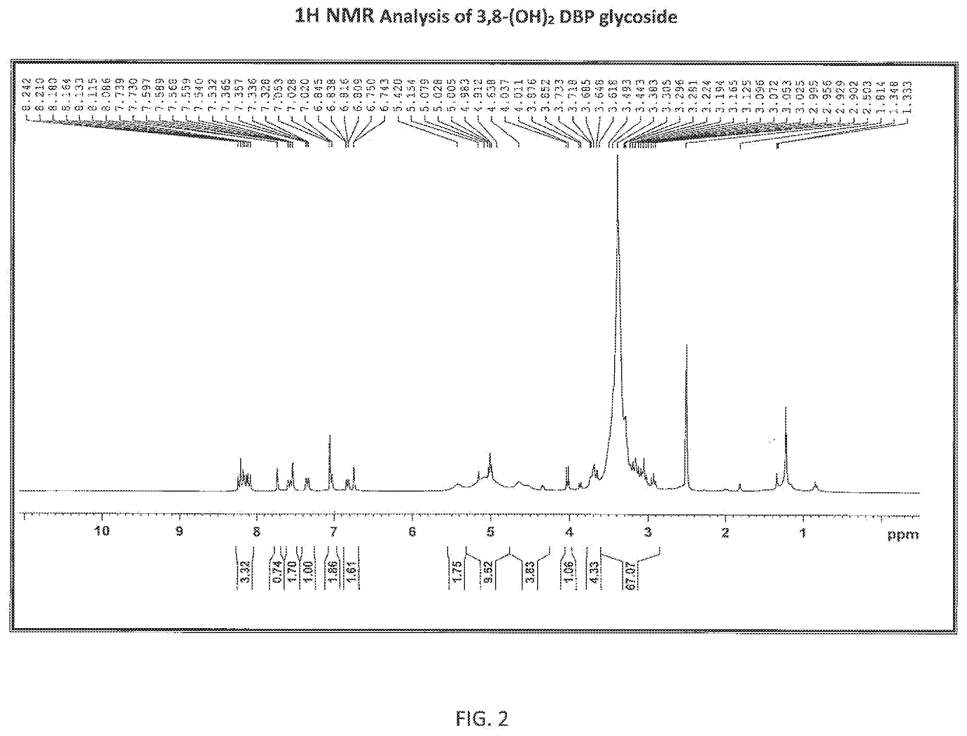

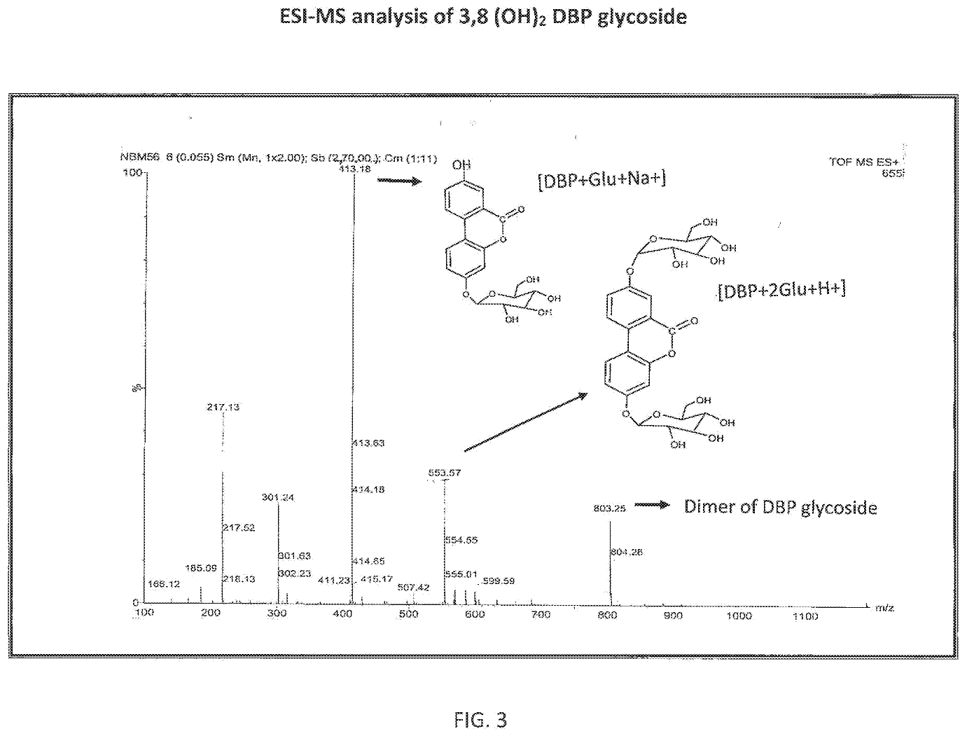

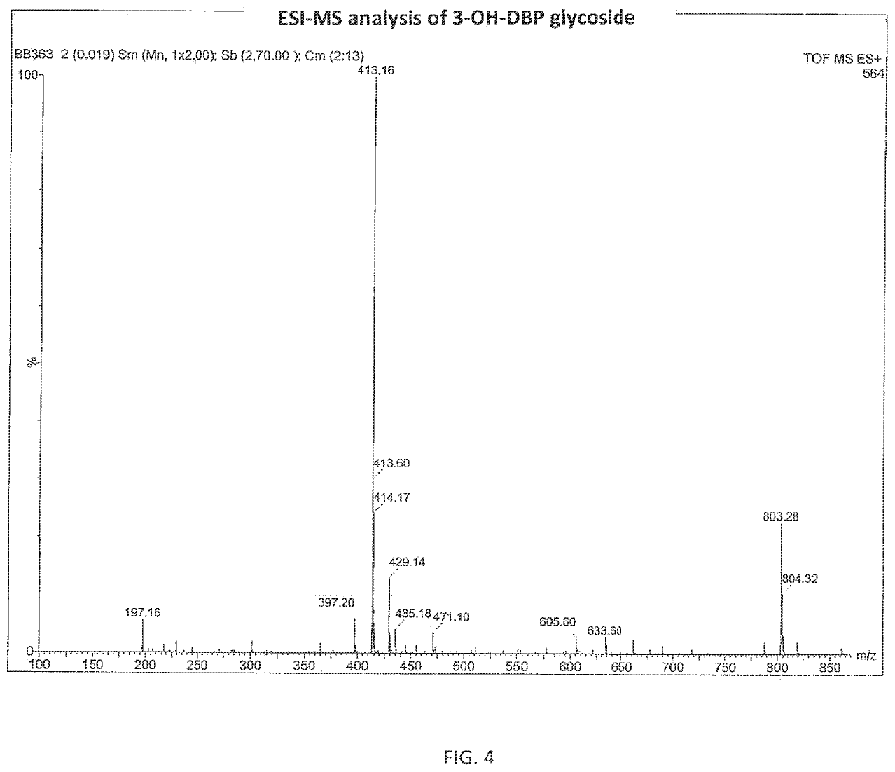

FIG. 1 shows the results of ESI-MS analysis of tetra-O-acetyl glycopyranosyl bromide according to this invention. The ESI-mass spectrum shows intensity, counts (y-axis, ranging from 0 to 3.9 e⁵) and m/z, amu (x-axis, ranging from 100 to 2000). Peaks shown are, left to right in ascending order: 109.0185, 113.9489, 129.0398, 169.0323, 239.0733, 271.0659, 301.1211, 331.0787, 353.0430, 371.0723, 387.0506, 413.0973, 434.9444, 450.9574, 463.2819, 492.9369, 685.4146, 709.3452, 750.3668, 797.1690, 844.9988, 902.9553. As noted on FIG. 1, the highest peak, 434.9444, indicates tetraacetylglucopyranosyl bromide (Mol. Wt. 411.2). To the right, the peak occurring at 844.9988 indicates a dimer of the substance. FIG. 2 shows the results of 1H NMR analysis of 3,8-(OH)₂ DBP glycoside of this invention, and is discussed in Example I. FIGS. 3 and 4 show the results of ESI-MS analysis of 3,8-(OH)₂ DBP glycoside (FIG. 3) and 3-(OH) DBP glycoside (FIG. 4), and are discussed in Example I. Peaks in FIG. 3 are identified at m/z 166.12, 185.09, 217.13, 217.52, 218.13, 301.24, 301.63, 302.23, 411.23, 413.18, 413.63, 414.18, 414.65, 415.17, 507.42, 553.57, 554.55, 555.01, 599.59, 803.25, 804.28. Peaks in FIG. 4 are identified at m/z 197.16, 397.20, 413.16, 413.60, 414.17, 429.14, 435.18, 471.10, 605.60, 633.60, 803.28, 804.32.

DBP glycosides may be synthesized under laboratory conditions by reacting 3-OH-DBP and/or 3,8-(OH)₂-DBP with tetra acetyl glucopyranosyl bromide. In an embodiment, DBP glycosides may then be purified by column chromatography, for instance to greater than 90% w/w purity, such as 90-95%, 95-99%, 97-99%, or other w/w purity or purity range. Purified DBP glycosides may be identified and characterized using spectroscopic (for instance 1H NMR and/or MASS) techniques.

In an embodiment, a process for preparing a DBP glycoside of the present invention comprises the step of reacting 3-OH-DBP and/or 3,8-(OH)₂-DBP with acetyl glucopyranosyl bromide such as tetra-O-acetyl glycopyranosyl bromide to provide the compound of Formula II (3-OH-DBP glycoside) and/or Formula III (3,8-(OH)₂-DBP glycoside). In an embodiment, a different bromide compound incorporating a different sugar such as a monosaccharide or disaccharide discussed above may be used. In an embodiment, said process of preparation further comprises the step of, prior to the bromination step, acetylating glucose such as D-glucose. In an embodiment, said process further comprises the step, after the bromination step, of purifying the DBP glycoside, for instance to 90-100% purity (w/w), including for instance 96-99% purity (w/w).

In an embodiment, a process for preparing a DBP glycoside according to the present invention comprises the steps of (a) acetylating a sugar, (b) brominating the acetylated sugar, and (c) glycosylating a DBP (i.e. adding the acetylated brominated sugar to a DBP to form a DBP glycoside). In an embodiment, the DBP glycoside is further purified for instance to 90-100 w/w purity, including for instance 96-99% w/w purity. In an embodiment, the sugar is glucose, xylose, rhamnose, arabinose, galactose, or a disaccharide containing glucose, xylose, rhamnose, arabinose, galactose. In an embodiment, the DBP glycoside prepared by this process is of Formula I, II, or III, as shown above.

A composition of the present invention may be formulated into nutraceutical or pharmaceutical dosage forms comprising for instance tablets, capsules, powders, liquids, chews, gummies, transdermals, injectables, dietary supplements, topical creams, lozenges, pills, suppositories, and so forth. A composition of the present invention may further comprise one or more excipients, additives, and/or other substances, including for instance microcrystalline cellulose, croscarmellose sodium, magnesium stearate, and/or silicon dioxide; and/or a suitable solution such as an aqueous buffer solution or carboxymethyl cellulose (CMC) solution. In an embodiment, a composition of the present invention may be made by combining 3-OH-DBP, 3-OH-DBP glycoside, 3,8-(OH)₂-DBP, and/or 3,8-(OH)₂-DBP glycoside with for instance microcrystalline cellulose, croscarmellose sodium, magnesium stearate, and/or silicon dioxide for instance through a simple mixing process or other suitable processes.

A “dietary supplement” according to the present invention refers to a composition of the present invention, comprising 3-OH-DBP, 3-OH-DBP glycoside, 3,8-(OH)₂-DBP, and/or 3,8-(OH)₂-DBP glycoside, which is orally administered as an addition to a subject's diet, which is not a natural or conventional food, which when administered delivers 3-OH-DBP, 3-OH-DBP glycoside, 3,8-(OH)₂-DBP, and/or 3,8-(OH)₂-DBP glycoside to the subject's blood and tissues and improves the immune system of the subject, and/or promotes immuno-protection. In an embodiment, the dietary supplement is administered daily. In an embodiment, the dietary supplement is administered daily for at least 1 day, 1 day to 1 week, 7 days, or 1 week to 4 weeks or to 8 weeks or to 12 weeks, or chronically for at least 3 months, 6 months, 9 months, or 1 year or more, or for another period of time according to the present invention. A dietary supplement may be formulated into various forms including a powder, as discussed throughout this application. In an embodiment, PrimaVie® Shilajit, in its original form or enriched with DBPs and/or DBP glycosides, is a dietary supplement of this invention.

“Administering”, “administration”, and the like, according to the present invention refer to providing a composition of the present invention to a subject so that the 3-OH-DBP, 3-OH-DBP glycoside, 3,8-(OH)₂-DBP, and/or 3,8-(OH)₂-DBP glycoside may reach the subject's blood and/or tissues; in an embodiment, in an amount effective for the 3-OH-DBP, 3-OH-DBP glycoside, 3,8-(OH)₂-DBP, and/or 3,8-(OH)₂-DBP glycoside to act on the subject's tissues, cells, blood, and/or other bodily components to improve the subject's immune system, provide immuno-protection, and/or treat immunosuppression. In an embodiment, the 3-OH-DBP, 3-OH-DBP glycoside, 3,8-(OH)₂-DBP, and/or 3,8-(OH)₂-DBP glycoside may be modified by the subject's body when administered to and/or acting on the subject's body.

In the present invention, an “effective amount” of 3-OH-DBP, 3-OH-DBP glycoside, 3,8-(OH)₂-DBP, and/or 3,8-(OH)₂-DBP glycoside, or “amount effective”, or the like, refers to an amount of 3-OH-DBP, 3-OH-DBP glycoside, 3,8-(OH)₂-DBP, and/or 3,8-(OH)₂-DBP glycoside needed to reach a subject's blood, tissues, cells, and/or other bodily components and to improve the subject's immune system, provide immuno-protection, and/or treat immunosuppression. In an embodiment, a composition of this invention includes an effective amount of 3-OH-DBP, 3-OH-DBP glycoside, 3,8-(OH)₂-DBP, and/or 3,8-(OH)₂-DBP glycoside, alone or in combination. An effective amount may be adjusted for different species according to this invention.

In an embodiment, an effective amount of 3-OH-DBP is a daily dosage of about 0.5 mg/kg body weight of a subject to about 75 mg/kg body weight. In an embodiment, an effective amount of 3-OH-DBP is a daily dosage of about 1 mg/kg body weight to about 50 mg/kg body weight or about 1.5 mg/kg to about 25 mg/kg. In an embodiment, an effective amount of 3-OH-DBP is a daily dosage of about 0.5 mg/kg, 2 mg/kg, 5 mg/kg, 10 mg/kg, 20 mg/kg, 25 mg/kg, 50 mg/kg, 75 mg/kg body weight of a subject. In an embodiment, in a human subject, an effective amount of 3-OH-DBP may include a daily dose ranging from 1 mg to 4000 mg, including for instance 50 mg, 100 mg, 200 mg, 250 mg, 400 mg, 500 mg, or 1000 mg daily.

In an embodiment, an effective amount of 3-OH-DBP glycoside is a daily dosage of about 0.5 mg/kg body weight of a subject to about 75 mg/kg body weight. In an embodiment, an effective amount of 3-OH-DBP glycoside is a daily dosage of about 1 mg/kg body weight to about 50 mg/kg body weight or about 1.5 mg/kg to about 25 mg/kg. In an embodiment, an effective amount of 3-OH-DBP glycoside is a daily dosage of about 0.5 mg/kg, 2 mg/kg, 5 mg/kg, 10 mg/kg, 20 mg/kg, 25 mg/kg, 50 mg/kg, 75 mg/kg, or in some cases higher, for instance 80 mg/kg, or 100 mg/kg body weight of a subject. In an embodiment, in a human subject, an effective amount of 3-OH-DBP glycoside may include a daily dose ranging from 1 mg to 4000 mg, including for instance 50 mg, 100 mg, 200 mg, 250 mg, 400 mg, 500 mg, or 1000 mg daily.

In an embodiment, an effective amount of 3,8-(OH)₂-DBP is a daily dosage of about 0.5 mg/kg body weight of a subject to about 75 mg/kg body weight, including for instance a daily dosage of about 1 mg/kg body weight to about 50 mg/kg body weight or about 1.5 mg/kg to about 25 mg/kg. In an embodiment, an effective amount of 3,8-(OH)₂-DBP is a daily dosage of about 5 mg/kg, 10 mg/kg, 25 mg/kg, 40 mg/kg, 50 mg/kg, 60 mg/kg, 75 mg/kg, or more, for instance about 100 mg/kg body weight of a subject. In an embodiment, in a human subject, an effective amount of 3,8-(OH)₂-DBP may include a daily dose ranging from 1 mg to 4000 mg, including for instance 50 mg, 100 mg, 200 mg, 250 mg, 400 mg, 500 mg, or 1000 mg daily.

In an embodiment, an effective amount of 3,8-(OH)₂-DBP glycoside is a daily dosage of about 0.5 mg/kg body weight of a subject to about 75 mg/kg body weight, including for instance a daily dosage of about 1 mg/kg body weight to about 50 mg/kg body weight or about 1.5 mg/kg to about 25 mg/kg. In an embodiment, an effective amount of 3,8-(OH)₂-DBP glycoside is a daily dosage of about 5 mg/kg, 10 mg/kg, 25 mg/kg, 40 mg/kg, 50 mg/kg, 60 mg/kg, 75 mg/kg, or 100 mg/kg body weight of a subject. In an embodiment, in a human subject, an effective amount of 3,8-(OH)₂-DBP glycoside may include a daily dose ranging from 1 mg to 4000 mg, including for instance 50 mg, 100 mg, 200 mg, 250 mg, 400 mg, 500 mg, or 1000 mg daily.

In an embodiment, combinations of 3-OH-DBP, 3-OH-DBP glycoside, 3,8-(OH)₂-DBP, and/or 3,8-(OH)₂-DBP glycoside, in any amounts or ratios for instance as described above and throughout this application, may be included in a composition of this invention. In an embodiment, a composition comprising 3-OH-DBP may include 3-OH-DBP alone (without other aforementioned DBPs and/or DBP glycosides), or 3-OH-DBP in combination with 3-OH-DBP glycoside, or 3-OH-DBP in combination with 3,8-(OH)₂-DBP, or 3-OH-DBP in combination with 3,8-(OH)₂-DBP glycoside; each of which may be further combined with one or more of the remaining DBPs identified above. Similarly, in an embodiment, a composition comprising 3-OH-DBP glycoside may include 3-OH-DBP glycoside alone (without other aforementioned DBPs and/or DBP glycosides), or 3-OH-DBP glycoside in combination with 3-OH-DBP, or 3-OH-DBP glycoside in combination with 3,8-(OH)₂-DBP, or 3-OH-DBP glycoside in combination with 3,8-(OH)₂-DBP glycoside; each of which may be further combined with one or more of the remaining DBPs identified above. Similarly, in an embodiment, a composition comprising 3,8-(OH)₂-DBP may include 3,8-(OH)₂-DBP alone (without other aforementioned DBPs and/or DBP glycosides), or 3,8-(OH)₂-DBP in combination with 3-OH-DBP, or 3,8-(OH)₂-DBP in combination with 3-OH-DBP glycoside, or 3,8-(OH)₂-DBP in combination with 3,8-(OH)₂-DBP glycoside; each of which may be further combined with one or more of the remaining DBPs identified above. Similarly, in an embodiment, a composition comprising 3,8-(OH)₂-DBP glycoside may include 3,8-(OH)₂-DBP glycoside alone (without other aforementioned DBPs and/or DBP glycosides), or 3,8-(OH)₂-DBP glycoside in combination with 3-OH-DBP, or 3,8-(OH)₂-DBP glycoside in combination with 3-OH-DBP glycoside, or 3,8-(OH)₂-DBP glycoside in combination with 3,8-(OH)₂-DBP; each of which may be further combined with one or more of the remaining DBPs and/or DBP glycosides identified above.

In an embodiment, an effective amount is a daily dosage of a combination of about 0.5-100 mg total DBP and/or DBP glycoside/kg body weight of a subject, including for instance 1-50 total mg/kg, or 1.5-25 mg total DBP and/or DBP glycoside/kg body weight. For instance, a total daily dosage may include about 0.5-100 mg 3-OH-DBP, 1-20 mg/kg, or about 2-10 mg/kg body weight of the subject and about 1-100 mg, 5-75 mg, or about 10-50 mg 3,8-(OH)₂-DBP/kg body weight of the subject. Compositions including an effective amount of combined 3-OH-DBP+3,8-(OH)₂-DBP are embodiments of this invention. In an embodiment, a synergistic composition of this invention comprises a synergistic combination of 3-OH-DBP, 3-OH-DBP glycoside, 3,8-(OH)₂-DBP, and/or 3,8-(OH)₂-DBP glycoside, where the components of the synergistic combination display an unexpected, greater effect than a single component alone. Amounts and ratios as discussed above this application may be used. Embodiments of synergistic compositions described in the below Examples are intended to be exemplary, and not limiting. In an embodiment, in a human subject, an effective amount of 3-OH-DBP, 3-OH-DBP glycoside, 3,8-(OH)₂-DBP, and/or 3,8-(OH)₂-DBP glycoside may include a daily dose, for each compound individually or combined together, ranging from 1 mg to 4000 mg, including for instance 50 mg, 100 mg, 200 mg, 250 mg, 400 mg, 500 mg, or 1000 mg daily.

Administration according to this invention may be by the subject or by another. Administration may be oral, for instance in the form of a dietary supplement, and/or in a solid dosage form, such as a capsule, and/or through other physiologically acceptable routes such as parenteral, intramuscular, transdermal, intraperitoneal, intravenous, rectal, and so forth.

A “subject” of the present invention refers to an animal having an immune system. In an embodiment, a subject is a mammal, such as a dog, cat, or horse. In a preferred embodiment, a subject is a human. In an embodiment, a subject is male; in another embodiment, a subject is female, according to this invention.

“Improving”, “improvement”, and the like of the immune system according to this invention refers to the action of 3-OH-DBP, 3-OH-DBP glycoside, 3,8-(OH)₂-DBP, and/or 3,8-(OH)₂-DBP glycoside at bodily tissues, blood, cells, and/or other bodily components of the subject, where said action for instance increases macrophage viability and/or activity, and/or increases splenocyte viability. In an embodiment, improvement of the immune system is evidenced by an increase (including a synergistic increase) in macrophage viability, macrophage activity, and/or splenocyte viability, for instance as shown in, but not limited to, the Examples below. In an embodiment, improvement of the immune system according to this invention refers to improvement of an immune system parameter that is suppressed or otherwise not functioning according to normal parameters for the individual and/or within a normal range for the individual's species, for instance due to an external influence such as an immunosuppressive substance (e.g. cyclophosphamide) or radiation including for instance chemotherapy or radiation therapy, and/or due to an impairment or dysfunction or other immunosuppression which results in a weakened or even inactive immune system and/or immune response in the subject. In an embodiment, parameters of the immune system include activity and/or viability of immune system components (such as macrophages and splenocytes), for instance macrophage viability, macrophage activity, and/or splenocyte viability. When immune system components and/or parameters are suppressed (i.e. lower than normal for the individual subject and/or on average for the subject's species), improvement in the immune system according to the present invention lies in increasing immune system parameters such as macrophage viability, macrophage activity, and/or splenocyte viability toward normal (for the individual or on average for the species) or even restoring the parameters to normal. See for instance some embodiments of this invention as described in the Examples. In an embodiment, suppressed parameters may be increased, restored, or even increased to levels higher than normal, for instance as seen in the below Examples. In an embodiment, the immune system is not stimulated to levels that may be or are harmful to the subject. In an embodiment, improving the immune system according to this invention may include improving immune response. In an embodiment, improvement of the immune system according to this invention refers to improvement of an immune system that is functioning within normal immune system parameters for the individual subject and/or on average for the subject's species, including but not limited to macrophage viability, macrophage activity, and/or splenocyte viability, said improvement nevertheless increasing immune system responsiveness or effectiveness, and increasing or otherwise improving one or more known immune system parameter. In an embodiment, improving the immune system according to this invention may include restoring one or more immune system parameters to normal or a normal range, for instance by increasing the parameters such as by increasing macrophage activity and/or viability and/or increasing splenocyte viability, or by decreasing immune system parameters to normal or a normal range, with compositions of this invention. In an embodiment, improving the immune system includes increasing immune system parameters that have decreased as the subject has aged and/or due to a poor diet and/or due to a disease such as an auto-immune disease. In an embodiment, improving the immune system includes decreasing immune system parameters that have increased as the subject has aged and/or due to a poor diet and/or due to a disease such as an auto-immune disease. In an embodiment, improving the immune system includes increasing and/or decreasing one or more immune system parameters to maintain a normal healthy immune system in the subject.

In an embodiment, suppression of the immune system or of the immune response may be treated according to the present invention by administering an effective amount of a composition including one or more of 3-OH-DBP, 3-OH-DBP glycoside, 3,8-(OH)₂-DBP, and 3,8-(OH)₂-DBP glycoside to a subject. In an embodiment, the 3-OH-DBP, 3-OH-DBP glycoside, 3,8-(OH)₂-DBP, and/or 3,8-(OH)₂-DBP glycoside will reach or be delivered to the subject's cells, tissues, and/or bloodstream and act upon the cells, tissues, and/or bloodstream to activate or otherwise stimulate one or more suppressed components of the immune system and/or immune response. In an embodiment, said treatment is a type of improvement of the immune system according to this invention. In an embodiment, said activation or other stimulation is evidenced by an increase in macrophage viability and/or activity, and/or by an increase in splenocyte activity, for instance as shown in the Examples. In an embodiment, said activation or other stimulation is evidenced by increases in other components or parameters of the immune system or immune response. In an embodiment, treatment of immunosuppression according to the present invention is described in the Examples below. In an embodiment, treatment of immunosuppression is of immunosuppression from cyclophosphamide or other drug treatment, substance, or physical state of the subject that may suppress immune system parameter(s), for instance in subjects having undergone organ transplantation where appropriate. An example of a successful treatment of suppression of the immune system is generating an increase in macrophage viability and/or activity and/or splenocyte viability.

In an embodiment, the administration of an effective amount of a composition of this invention to a subject (including the DBPs/DBP glycosides therein) provides immuno-protection to the subject, for instance, by improving or maintaining the subject's immune response to foreign or abnormal antigens by improving or maintaining macrophage viability and/or activity and/or splenocyte viability. In an embodiment, said immuno-protection is of a normal healthy immune system, with one or more immune system parameters including but not limited to macrophage viability and/or activity and/or splenocyte viability maintained in a range normal for the subject or for an average member of the subject's species. In an embodiment, said immuno-protection is of a suppressed immune system, with one or more immune system parameters including but not limited to macrophage viability and/or activity and/or splenocyte viability in a range that is abnormal for the subject or for an average member of the subject's species, and wherein said abnormal range indicates a suppressed (less active than normal) immune system. In an embodiment, immunoprotection refers to administering a composition of the present invention and thereby maintaining a subject's immune system parameters in the presence of an immunosuppressive condition such as an immunosuppressing substance (e.g. immunosuppressant substance such as cyclophosphamide) or physical state (e.g. ill health, age, poor diet), such as discussed throughout this application.

The present invention may be further understood in connection with the following Examples and embodiments. The Examples and embodiments described throughout this application are provided to illustrate the invention and are not intended as limiting.

DBP glycosides 3-O-Glucopyranosyl-Dibenzo-alpha-pyrone (3-OH-DBP glycoside, 3-OH-DBP glucoside) and 3,8-di-(O-Glucopyranosyl)-Dibenzo-alpha-pyrone (3,8-(OH)₂-DBP glycoside, 3,8-(OH)₂-DBP glucoside) were synthesized under laboratory conditions by reacting 3-OH-DBP and/or 3,8-(OH)₂-DBP with tetra acetyl glucopyranosyl bromide. The resulting glycosides were purified by column chromatography and 98% pure DBP glycosides (3-OH-DBP glucoside and 3,8-(OH)₂-DBP glucoside) were obtained. Purified DBP glycosides (3-OH-DBP glucoside and 3,8-(OH)₂-DBP glucoside) were identified and characterized using spectroscopic (for instance 1H NMR and/or MASS) techniques. 3-OH-DBP glucoside and 3,8-(OH)₂-DBP glucoside, also referred to as 3-OH-DBP glycoside and 3,8-(OH)₂-DBP glycoside (without being limiting), were used in the below Examples.

### Example I

DBP glycosides according to this invention were prepared in three steps: Acetylation of D-glucose, Bromination of the acetyl glucopyranoside, and then Glycosylation of DBPs.

### I. Acetylation of D-glucose

Acetic anhydride (25 ml, 260 mM) was added to a mixture of finely ground anhydrous sodium acetate (4.0 g, 48.8 mM) and D-glucose (5.0 g, 28 mM). The mixture was heated at 70° C. with occasional stirring until a clear solution was obtained. The heating was continued for 10 hours at the same temperature. The reaction mixture was poured on ice water (250 ml) with stirring and gave a white solid, the solid was filtered off and further used for the bromination reaction as described below. The formation of product was confirmed by mass spectrum. Yield=85.93%.

### II. Bromination of Penta-O-Acetyl Glucopyranoside

1, 2, 3, 4, 6-penta-O-acetyl-D-glucopyranose was added (10 g, 24 mM) portion wise (0.5 g at a time) to a stirred solution of HBr (33%) in glacial acetic acid (25 ml) at 0° C. After all the sugar was added, the reaction mixture was further cooled for 45 minutes. TLC analysis Hexane:Ethyl acetate (1:1) indicated formation of product tetra-O-acetyl-glucopyranoside bromide (R_{f}=0.5). The reaction was quenched with ice water (50 ml). The precipitate was filtered and dried. The formation of product was confirmed by mass spectrum as discussed below. Yield=53.33%.

### ESI-TOF Mass Spectrometry

The ESI-TOF mass spectrometry of Tetra-O-acetyl glucopyranosyl bromide was performed using the Applied Biosystems Sciex QSTAR XL MS/MS System (ThermoFisher Scientific, Waltham Mass., USA) (FIG. 1). The mass spectrum exhibited a molecular ion peak at m/z 434.18 [M+Na]⁺ and 844 (dimer [M+Na]⁺) mass which is in corroboration with the molecular mass of Tetra-O-Acetyl Glucopyranosyl Bromide.

### III. Glycosylation of DBP's

1) 3,8-(OH)₂-DBP (500 mg) was added into an ice cold solution of 10 ml of methanolic KOH (potassium hydroxide) and stirred for half an hour.

2) In another 50 ml RBF (round-bottom flask), (940 mg) tetra-O-acetyl glucopyranosyl bromide was dissolved in 10 ml of acetone, and the resulting reaction mixture was cooled.

3) To the tetra-acetyl glucopyranosyl bromide in acetone, the ice cold mixture of DBP in methanolic KOH was added drop wise over the period of half an hour, and the entire aliquot was further cooled at 0° C. for 6 hours and left at room temperature overnight. The entire reaction was carried out under N₂ atmosphere.

4) Work-up procedure: Water was added to quench the reaction, methanol and acetone were evaporated and the reaction mixture partitioned with ethyl acetate to remove organic impurities.

5) Column chromatography was done for purification of 3,8-(OH)₂-DBP glycoside. Yield was found to be 40-45%.

6) The same procedure was followed for Glycosylation of 3-OH-DBP.

¹H NMR analysis of 3,8 (OH)₂ DBP glycoside

3,8-(OH)₂-DBP marker was solubilized in deuterio-water and subjected into (300 MHz) ¹H-NMR analysis (Bruker-Avance II 300 MHz Ultrashield). The ¹H-NMR spectrum (FIG. 2) of compound exhibited signals in the region δ6.5-8.3 ppm corresponding to six aromatic protons of 3,8-(OH)₂-DBP. Signal at δ4.98 ppm is characteristic of anomeric proton of glycoside.

ESI-TOF mass spectrometry

ESI-TOF mass spectrometry of 3,8-(OH)₂-DBP glycoside was performed using Applied Biosystems Sciex QSTAR XL MS/MS System (ThermoFisher Scientific, Waltham Mass., USA) (FIG. 3). The mass spectrum exhibited a molecular ion peak at m/z 413.18 [M+Na]⁺ [DBP+Glucose+Na⁺], 553 [DBP+2Glucose+H⁺], and 803 (Dimer of [M+Na]⁺ DBP glycoside) mass, as shown in FIG. 3, which is in corroboration with the molecular mass of 3,8-(OH)₂-DBP glycoside.

ESI-TOF mass spectrometry of 3-OH-DBP glycoside was performed using Applied Biosystems Sciex QSTAR XL MS/MS System (ThermoFisher Scientific, Waltham Mass., USA) (FIG. 4). The mass spectrum exhibited a molecular ion peak at m/z 413.16 [M+Na]⁺ and 803 (dimer of [M+Na]⁺ DBP glycoside) mass which is in keeping with the molecular mass of 3-OH-DBP glycoside.

### Example II

The below Example shows improved immune system function with the administration of 3-OH-DBP glycoside (10 mg/kg body weight) and 3,8-(OH)₂-DBP glycoside (10 mg/kg body weight) in cyclophosphamide-induced immunosuppression.

### Materials and Methods

Test compound 3-OH-DBP (Natreon Inc., New Brunswick, N.J.) was characterized as a white free-flowing powder.

Test compound 3-OH-DBP glycoside (Natreon Inc., New Brunswick, N.J.) was characterized as a light brown free-flowing powder.

Test compound 3,8-(OH)₂-DBP (Natreon Inc.) was characterized as a light yellow free-flowing powder.

Test compound 3,8-(OH)₂-DBP glycoside (Natreon Inc.) was characterized as a brown free-flowing powder.

Cyclophosphamide (Sigma, Batch No. 084K1328) was characterized as a white free-flowing powder.

Experimental Animals:

Swiss albino mice of both sexes, weighing approximately 25-30 g, were obtained from National Research Institute of Ayurveda for Drug Development (Govt. of India), Kolkata, and were housed in polypropylene cages at 22±3° C., relative air humidity of 45 to 55%, with 12.00 hr light and dark cycle (lighting on from 6:00 AM to 6:00 PM) and were provided standard pellet chow (carbohydrate 65.5%, protein 17.6%, fat 6.6%) and distilled water ad libitum. The mice were acclimatized for one week in the laboratory conditions, before being used in the experiment. All experiments were conducted between 10.00 hr and 14.00 hr. Principles of laboratory animal care (NIH publication no. 85-23, revised 1985) were followed.

Drug Protocol:

The animals were randomly assigned into six Groups of six animals each and were administered compounds as shown in Table 1.

TABLE 1
Animal Groups, Controls, and Treatments

| Group | Control/Treatment |
|---|---|
| I | Vehicle control (0.3% CMC, p.o) |
| II | Cyclophosphamide (250 mg/kg body weight, i.p) |
| III | 3-OH-DBP (10 mg/kg body weight, p.o.) + |
| | Cyclophosphamide (250 mg/kg body weight, i.p) |
| IV | 3,8-(OH)₂-DBP (10 mg/kg body weight, p.o.) + |
| | Cyclophosphamide (250 mg/kg body weight, i.p) |
| V | 3-OH-DBP glycoside (10 mg/kg body weight, p.o.) + |
| | Cyclophosphamide (250 mg/kg body weight, i.p.) |
| VI | 3,8-(OH)₂-DBP glycoside (10 mg/kg body weight, p.o.) + |
| | Cyclophosphamide (250 mg/kg body weight, i.p.) |

Regarding Group II, see Singh S, Yadav C P S, Noolvi M N, “Immunomodulatory activity of butanol fraction of Gentiana olivieri Griseb. on Balb/C mice” Asian Pac J Trop Biomed. 2(6): 433-437 (2012).

All test compounds (DBPs and DBP glycosides) were suspended in 0.3% of CMC (Carboxymethyl Cellulose) solutions of distilled water and were administered orally for 7 days by using an intubation canula. The volume of dose was 100 μl/10 g body weight. Cyclophosphamide was dissolved in distilled water and was injected (250 mg/kg, i.p.) on day one and volume of dose was 100 μl/10 g body weight. Control animals received equivalent volume of the vehicles CMC (0.3%) solutions only.

Isolation of Peritoneal Macrophages:

On day seven all the animals were sacrificed under ether anesthesia and peritoneal macrophages were lavaged using ice cold phosphate buffer saline (PBS: 0.15M NaCl pH-7.4). After centrifugation (3000 rpm×10 min) at 4° C. the pellet was re-suspended in PBS and cell viability was confirmed by trypan blue exclusion method. (Bala A, Haldar P K, Kar B, Naskar S and Mazumder U K, “Carbon tetrachloride: A hepatotoxin causes oxidative stress in murine peritoneal macrophage and peripheral blood lymphocyte cells” Immunopharmacol. and Immunotoxicol. 34(1): 157-162 (2012)).

Isolation of Splenocytes:

Spleen was collected. Single cell suspension was prepared by mincing and tapping the spleen fragments on stainless steel 200-mesh in ice cold phosphate buffer saline (PBS: 0.15M NaCl pH 7.4). Cell suspension was centrifuged at 2000 rpm for 10 minutes. The pellet was re-suspended in PBS and cell viability was confirmed by trypan blue exclusion method.

NBT Reduction Assay:

Preparation of NBT solution: 0.2% NBT (nitroblue tetrazolium) in PB (Phosphate Buffer, pH 7.4), 5% dextrose and HBSS (Hank's balanced salt solution). The three solutions were mixed in 6:2:4 ratios. (Saxena A K, Singh K P, Srivastava S N, Shukla L J and Shanker R., “Immunomodulating effects of caffeine in rodents” Ind J Exptl Biol. 22: 298-301 (1984)).

Method: 0.5 ml of NBT solution was added to 0.5 ml of peritoneal macrophages (1×10⁶ cells/ml) in a micro-centrifuge tube. This mixture was incubated at 37° C. for 45 minutes. The cell suspension was centrifuged at 1500 rpm for 10 minutes. Supernatant was discarded and pellet was re-suspended in 2 ml of DMSO. The tubes were kept in a boiling water bath for 10 minutes. The tubes were again centrifuged at 1500 rpm for 10 minutes. The supernatant was collected, and absorbance was measured at 515 nm.

### Results and Discussion

The NBT reduction test is an indirect marker of oxygen-dependent bactericidal activity of peritoneal macrophages. The present results indicate that cyclophosphamide suppressed the immune function of peritoneal macrophages as evidenced by a significant (p<0.001) decrease in NBT reduction capacity (Table 2). Treatment with 3-OH-DBP glycoside at the dose level of 10 mg/kg body weight (p.o.) showed highest level of improvement (p<0.001) in the NBT reduction capacity in comparison with cyclophosphamide treated mice among all the treatment groups; whereas treatment with 3-OH-DBP and 3,8-(OH)₂-DBP glycoside demonstrated moderate (p<0.05) improvement in NBT reduction capacity (Table 2).

TABLE 2
NBT reduction assay in peritoneal macrophages

| Group | OD (515 nm) |
|---|---|
| CMC control (0.3% CMC) | 0.233 ± 0.034 |
| Cyclophosphamide | 0.117 ± 0.015*** |
| 3-OH-DBP + Cyclophosphamide | 0.154 ± 0.019^{#} |
| 3,8-(OH)₂-DBP + Cyclophosphamide | 0.144 ± 0.010 |
| 3-OH-DBP glycoside + Cyclophosphamide | 0.168 ± 0.014^{##} |
| 3,8-(OH)₂-DBP glycoside + Cyclophosphamide | 0.153 ± 0.013^{#} |

Data are expressed as Mean ± SD, n = 6.
p value was obtained by ANOVA followed by post hoc comparison by Bonferroni's multiple comparison test.
***p < 0.001 in comparison to CMC control mice.
^{##}p < 0.01,
^{#}p < 0.05 in comparison to cyclophosphamide treated mice

The administration of cyclophosphamide significantly (p<0.01) decreased splenocyte viability (Table 3). A significant improvement in splenocyte viability was observed after treatment with 3-OH-DBP glycoside in cyclophosphamide-treated mice.

TABLE 3
Cell viability of splenocytes

| Group | % Cell Viability |
|---|---|
| CMC control (0.3% CMC) | 69.31 ± 4.24 |
| Cyclophosphamide | 51.12 ± 4.69*** |
| 3-OH-DBP + Cyclophosphamide | 59.70 ± 3.59 |
| 3,8-(OH)₂-DBP + Cyclophosphamide | 54.90 ± 5.01 |
| 3-OH-DBP glycoside + Cyclophosphamide | 60.39 ± 5.85^{#} |
| 3,8-(OH)₂-DBP glycoside + Cyclophosphamide | 58.58 ± 4.49 |

Data are expressed as Mean ± SD, n = 6.
p value was obtained by ANOVA followed by post hoc comparison by Bonferroni's multiple comparison test.
***p < 0.001 in comparison to CMC control mice.
^{#}p < 0.05 in comparison to cyclophosphamide treated mice.

### Conclusion

Cyclophosphamide-induced immunosuppression was rescued with the treatment of 3-OH-DBP glycoside and 3,8-(OH)₂-DBP glycoside for 7 days at the dose level of 10 mg/kg body weight (p.o.).

### Example III

This Example shows that 3-OH-DBP, 3,8-(OH)₂-DBP and combined 3-OH-DBP+3,8-(OH)₂-DBP (1:5 ratio) improve the immune system in cyclophosphamide-induced immunosuppression of male and female mice.

### Materials and Methods

Test compound 3-OH-DBP (Natreon Inc., New Brunswick, N.J.) was characterized as a white free-flowing powder.

Test compound 3,8-(OH)₂-DBP (Natreon Inc.) was characterized as a green free-flowing powder.

Test compound 3-OH-DBP+3,8-(OH)₂-DBP combined (1:5 ratio w/w) was characterized as a light green free-flowing powder.

Experimental Animals:

Male and female Swiss albino mice (n=8; 4 from each gender) weighing approximately 25±5 g, were obtained from National Research Institute of Ayurveda for Drug Development (Govt. of India), Kolkata, and were housed in polypropylene cages at 22±3° C., relative air humidity of 45 to 55%, with 12.00 h light & dark cycle (lighting on from 6:00 AM to 6:00 PM). Mice were provided a standard pellet chow diet (carbohydrate 65.5%, protein 17.6%, fat 6.6%) and distilled water ad libitum. The mice were acclimatized for one week in the laboratory conditions, before being used in the experiment. All experiments were conducted between 10.00 h and 14.00 h. Principles of laboratory animal care (NIH publication no. 85-23, revised 1985) was followed.

Experimental Procedure and Drug Regime

Induction of Immunosuppression

Cyclophosphamide was dissolved in distilled water and the mice were administered a single exposure (250 mg/kg, i.p.) on day 1.

Treatment Schedule

3-OH-DBP, 3,8-(OH)₂-DBP, and combined 3-OH-DBP+3,8-(OH)₂-DBP were administered once daily for 7 days starting from day 1. Following 7 days of treatment the mice were anaesthetized and sacrificed under anaesthesia followed by collection of peritoneal macrophages and spleen (for splenocyte isolation).

Drug Regime

Swiss albino mice were randomly divided into 8 Groups (n=8; 4 from each gender). As shown in Table 4, Group I animals served as vehicle control and received 0.3% CMC, Group II received cyclophosphamide (250 mg/kg b. w., i.p., single exposure), Group III mice were administered 3-OH-DBP (2 mg/kg b.w., p.o.), Group IV mice were administered 3,8-(OH)₂-DBP (10 mg/kg b.w., p.o.), Group V mice were administered [3-OH-DBP+3,8-(OH)₂-DBP] (2:10) (12 mg/kg b.w., p.o.), Group VI mice were administered 3-OH-DBP (10 mg/kg b.w., p.o.), Group VII mice were administered 3,8-(OH)₂-DBP (50 mg/kg b.w., p.o.), Group VIII mice were administered [3-OH-DBP+3,8-(OH)₂-DBP] (10:50) (60 mg/kg b.w., p.o.).

TABLE 4
Animal Groups, Controls, and Treatments

| Group | Control/Treatment |
|---|---|
| I | Control Vehicle (0.3% CMC) |
| II | Cyclophosphamide (250 mg/kg b.w., i.p.) |
| III | Cyclophosphamide (250 mg/kg b.w., i.p.) + |
| | 3-OH-DBP (2 mg/kg b.w., p.o.) |
| IV | Cyclophosphamide (250 mg/kg b.w., i.p.) + |
| | 3,8-(OH)₂-DBP (10 mg/kg b.w., p.o.) |
| V | Cyclophosphamide (250 mg/kg b.w., i.p.) + |
| | [3-OH-DBP 2 mg + 3,8-(OH)₂-DBP 10 mg] |
| | (12 mg/kg b.w., p.o.) |
| VI | Cyclophosphamide (250 mg/kg b.w., i.p.) + |
| | 3-OH-DBP (10 mg/kg b.w., p.o.) |
| VII | Cyclophosphamide (250 mg/kg b.w., i.p.) + |
| | 3,8-(OH)₂-DBP (50 mg/kg b.w., p.o.) |
| VIII | Cyclophosphamide (250 mg/kg b.w., i.p.) + |
| | [3-OH-DBP 10 mg + 3,8-(OH)₂-DBP 50 mg] |
| | (60 mg/kg b.w., p.o.) |

Experimental Protocol

Isolation of Peritoneal Macrophages:

All the animals were sacrificed under ether anesthesia and peritoneal macrophages were lavaged using ice cold phosphate buffer saline (PBS: 0.15 M NaCl; pH: 7.4). After centrifugation (3000 rpm×10 min) at 4° C., pellets were re-suspended in PBS and cell viability was confirmed by trypan blue exclusion assay.

Isolation of Splenocytes:

Spleens were collected and single cell suspension was prepared by mincing and tapping the spleen fragments on stainless steel 200-mesh in ice cold phosphate buffer saline (PBS: 0.15M NaCl; pH: 7.4). Cell suspensions were centrifuged at 2000 rpm for 10 minutes. The pellet was resuspended in PBS and cell viability was confirmed by trypan blue exclusion assay.

NBT Reduction Assay:

Preparation of NBT solution: 0.2% NBT (nitroblue tetrazolium) in PBS (pH 7.4), 5% dextrose and HBSS. These three solutions were mixed in 6:2:4 ratios.

0.5 ml of NBT solution was added to 0.5 ml of peritoneal macrophages (1×10⁶ cells/ml) in micro-centrifuge tube. This mixture was incubated at 37° C. for 2 hours. The cell suspension was centrifuged at 2000 rpm for 10 minutes. Supernatants were discarded and pellets were re-suspended in 1 ml of DMSO. The tubes were kept in boiling water bath for 10 minutes and then cooled and centrifuged at 2000 rpm for 10 minutes. The supernatants were collected and absorbance was measured at 515 nm.

Statistical Analysis:

Results were expressed in terms of mean±SEM (n=8; 4 from each gender). Data were subjected to one way ANOVA followed by Tukey's test using GraphPad Prism 4.0 software to establish statistical significance (*p<0.05, **p<0.01, ***p<0.001, where * refers to comparison with Group I vehicle control; #p<0.05, ##p<0.01, ###p<0.001, where #refers to comparison with Group II cyclophosphamide administration; ^{a}p<0.05, ^{aa}p<0.01, ^{aa}p<0.01, where a refers to comparison of Day 7 with Day 0).

### Results

Effects of 3-OH-DBP, 3,8-(OH)₂-DBP, and 3-OH-DBP+3,8-(OH)₂-DBP on the body weight, feed intake, water intake, macrophage viability, NBT reduction, and splenocyte viability on cyclophosphamide-induced immunosuppression in male and female mice are discussed below.

TABLE 5
Effect of 3-OH-DBP, 3,8-(OH)₂-DBP, and combined 3-
OH-DBP + 3,8-(OH)₂-DBP on body weight of male mice

| | | Body Weight (gm) | |
|---|---|---|---|
| Group | Treatment | Day 0 | Day 7 |
| I | Vehicle Control (0.3% CMC) | 28.00 ± 1.29 | 30.50 ± 2.02 |
| II | Cyclophosphamide (250 mg/kg, b.w.) | 30.75 ± 1.89 | 30.50 ± 2.18 |
| III | Cyclophosphamide (250 mg/kg, b.w.) + | 25.25 ± 1.44 | 24.50 ± 1.26 |
| | 3-OH-DBP (2 mg/kg, b.w.) | | |
| IV | Cyclophosphamide (250 mg/kg, b.w.) + | 28.00 ± 0.71 | 27.25 ± 0.63 |
| | 3,8-(OH)₂-DBP (10 mg/kg, b.w.) | | |
| V | Cyclophosphamide (250 mg/kg, b.w.) + | 27.75 ± 1.32 | 25.50 ± 1.76 |
| | 3-OH-DBP + 3,8-(OH)₂-DBP (12 mg/kg, b.w.) | | |
| VI | Cyclophosphamide (250 mg/kg, b.w.) + | 23.00 ± 1.29 | 22.75 ± 1.18 |
| | 3-OH-DBP (10 mg/kg, b.w.) | | |
| VII | Cyclophosphamide (250 mg/kg, b.w.) + | 28.50 ± 1.26 | 28.25 ± 1.75 |
| | 3,8-(OH)₂-DBP (50 mg/kg, b.w.) | | |
| VIII | Cyclophosphamide (250 mg/kg, b.w.) + | 25.50 ± 2.18 | 25 ± 1.35 |
| | 3-OH-DBP + 3,8-(OH)₂-DBP (60 mg/kg, b.w.) | | |

Body weight of male 3-OH-DBP, 3,8-(OH)₂-DBP, and combined 3-OH-DBP+3,8-(OH)₂-DBP treated mice were compared with vehicle control and cyclophosphamide treated male mice. See Table 5.

No significant (p>0.05) change in body weight was observed in the male mice.

TABLE 6
Effect of 3-OH-DBP, 3,8-(OH)₂-DBP, and combined
3-OH-DBP + 3,8-(OH)₂-DBP on body weight of female mice

| | | Body Weight (gm) | |
|---|---|---|---|
| Group | Treatment | Day 0 | Day 7 |
| I | Vehicle Control (0.3% CMC) | 23.00 ± 1.08 | 24.25 ± 0.85 |
| II | Cyclophosphamide (250 mg/kg, b.w.) | 23.00 ± 1.89 | 21.75 ± 0.63 |
| III | Cyclophosphamide (250 mg/kg, b.w.) + | 23.75 ± 1.85 | 23.25 ± 1.60 |
| | 3-OH-DBP (2 mg/kg, b.w.) | | |
| IV | Cyclophosphamide (250 mg/kg, b.w.) + | 22.50 ± 1.85 | 20.50 ± 1.50 |
| | 3,8-(OH)₂-DBP (10 mg/kg, b.w.) | | |
| V | Cyclophosphamide (250 mg/kg, b.w.) + | 22.50 ± 0.50 | 20.25 ± 1.44 |
| | 3-OH-DBP + 3,8-(OH)₂-DBP (12 mg/kg, b.w.) | | |
| VI | Cyclophosphamide (250 mg/kg, b.w.) + | 23.00 ± 0.58 | 21.75 ± 0.25 |
| | 3-OH-DBP (10 mg/kg, b.w.) | | |
| VII | Cyclophosphamide (250 mg/kg, b.w.) + | 22.00 ± 0.82 | 19.50 ± 0.65^{a} |
| | 3,8-(OH)₂-DBP (50 mg/kg, b.w.) | | |
| VIII | Cyclophosphamide (250 mg/kg, b.w.) + | 22.25 ± 0.85 | 21 ± 1.08 |
| | 3-OH-DBP + 3,8-(OH)₂-DBP (60 mg/kg, b.w.) | | |

Body weight of female 3-OH-DBP, 3,8-(OH)₂-DBP, and combined 3-OH-DBP+3,8-(OH)₂-DBP treated mice were compared with vehicle control and cyclophosphamide treated female mice.

As shown in Table 6, body weight was significantly (p<0.05) decreased on Day 7 in comparison to Day 0 in 3,8-(OH)₂-DBP (50 mg/kg, b.w.) treated female mice.

TABLE 7
Effect of 3-OH-DBP, 3,8-(OH)₂-DBP, and combined 3-
OH-DBP + 3,8-(OH)₂-DBP on feed intake of male mice

| | | Feed Intake (gm) | |
|---|---|---|---|
| Group | Treatment | Day 0 | Day 7 |
| I | Vehicle Control (0.3% CMC) | 29.00 ± 0.41 | 28.25 ± 0.85 |
| II | Cyclophosphamide (250 mg/kg, b.w.) | 28.50 ± 0.96 | 26.81 ± 0.86^{a} |
| III | Cyclophosphamide (250 mg/kg, b.w.) + | 28.25 ± 1.44 | 28.25 ± 1.03 |
| | 3-OH-DBP (2 mg/kg, b.w.) | | |
| IV | Cyclophosphamide (250 mg/kg, b.w.) + | 28.75 ± 0.95 | 22.75 ± 0.48^{aa} |
| | 3,8-(OH)₂-DBP (10 mg/kg, b.w.) | | |
| V | Cyclophosphamide (250 mg/kg, b.w.) + | 28.50 ± 0.65 | 28.25 ± 1.03 |
| | 3-OH-DBP + 3,8-(OH)₂-DBP (12 mg/kg, b.w.) | | |
| VI | Cyclophosphamide (250 mg/kg, b.w.) + | 29.75 ± 0.25 | 19.25 ± 1.25***,^{aaa} |
| | 3-OH-DBP (10 mg/kg, b.w.) | | |
| VII | Cyclophosphamide (250 mg/kg, b.w.) + | 28.50 ± 0.87 | 25.25 ± 1.38*,^{a} |
| | 3,8-(OH)₂-DBP (50 mg/kg, b.w.) | | |
| VIII | Cyclophosphamide (250 mg/kg, b.w.) + | 27.75 ± 0.85 | 28 ± 1.41 |
| | 3-OH-DBP + 3,8-(OH)₂-DBP (60 mg/kg, | | |
| | b.w.) | | |

Feed intake of male 3-OH-DBP, 3,8-(OH)₂-DBP, and combined 3-OH-DBP+3,8-(OH)₂-DBP treated mice was compared with vehicle control and cyclophosphamide treated male mice.

As shown in Table 7, feed intake was significantly (p<0.05, p<0.01) decreased in male cyclophosphamide, 3-OH-DBP (10 mg/kg, b.w), and 3,8-(OH)₂-DBP (50 mg/kg, b.w) treated mice when compared to vehicle control male mice. Feed intake was significantly (p<0.05, p<0.01) decreased in male cyclophosphamide, 3-OH-DBP (10 mg/kg, b.w), and 3,8-(OH)₂-DBP (50 mg/kg, b.w) treated mice when compared to vehicle control male mice. Feed intake was significantly (p<0.05, p<0.01) decreased in male cyclophosphamide, 3-OH-DBP (10 mg/kg, b.w) and 3,8-(OH)₂-DBP (10 and 50 mg/kg, b.w) treated mice on Day 7 in comparison to Day 0.

TABLE 8
Effect of 3-OH-DBP, 3,8-(OH)₂-DBP, and combined
3-OH-DBP + 3,8-(OH)₂-DBP on feed intake of female mice

| | | Feed Intake (gm) | |
|---|---|---|---|
| Group | Treatment | Day 0 | Day 7 |
| I | Vehicle Control (0.3% CMC) | 29.25 ± 0.48 | 28.31 ± 0.47 |
| II | Cyclophosphamide (250 mg/kg, b.w.) | 28.50 ± 0.96 | 24.71 ± 0.41^{a} |
| III | Cyclophosphamide (250 mg/kg, b.w.) + | 28.50 ± 1.19 | 29.06 ± 0.41 |
| | 3-OH-DBP (2 mg/kg, b.w.) | | |
| IV | Cyclophosphamide (250 mg/kg, b.w.) + | 28.25 ± 1.03 | 25.23 ± 0.47^{a} |
| | 3,8-(OH)₂-DBP (10 mg/kg, b.w.) | | |
| V | Cyclophosphamide (250 mg/kg, b.w.) + | 28.25 ± 1.18 | 27 ± 0.41 |
| | 3-OH-DBP + 3,8-(OH)₂-DBP (12 mg/kg, b.w.) | | |
| VI | Cyclophosphamide (250 mg/kg, b.w.) + | 29.00 ± 0.71 | 20.50 ± 0.65***^{,aaa} |
| | 3-OH-DBP (10 mg/kg, b.w.) | | |
| VI | Cyclophosphamide (250 mg/kg, b.w.) + | 27.75 ± 1.13 | 19.19 ± 0.28***^{,aaa} |
| | 3,8-(OH)₂-DBP (50 mg/kg, b.w.) | | |
| VIII | Cyclophosphamide (250 mg/kg, b.w.) + | 29.25 ± 0.48 | 27.50 ± 0.65 |
| | 3-OH-DBP + 3,8-(OH)₂-DBP (60 mg/kg, b.w.) | | |

Feed intake of female 3-OH-DBP, 3,8-(OH)₂-DBP, and combined 3-OH-DBP+3,8-(OH)₂-DBP treated mice was compared with vehicle control and cyclophosphamide treated female mice.

As shown in Table 8, feed intake was significantly (p<0.05, p<0.01) decreased in female cyclophosphamide, 3-OH-DBP (10 mg/kg, b.w), and 3,8-(OH)₂-DBP (50 mg/kg, b.w) treated mice when compared to vehicle control female mice. Feed intake was significantly (p<0.05, p<0.01) decreased in female 3-OH-DBP (10 mg/kg, b.w) and 3,8-(OH)₂-DBP (50 mg/kg, b.w) treated mice on Day 7 in comparison to Day 0.

TABLE 9
Effect of 3-OH-DBP, 3,8-(OH)₂-DBP, and combined
3-OH-DBP + 3,8-(OH)₂-DBP on water intake of male mice

| | | Water Intake (ml) | |
|---|---|---|---|
| Group | Treatment | Day 0 | Day 7 |
| I | Vehicle Control (0.3% CMC) | 5.30 ± 0.24 | 4.813 ± 0.21 |
| II | Cyclophosphamide (250 mg/kg, b.w.) | 6.125 ± 0.16 | 5.863 ± 0.15*,## |
| III | Cyclophosphamide (250 mg/kg, b.w.) + | 2.875 ± 0.16***,### | 2.875 ± 0.16***,### |
| | 3-OH-DBP (2 mg/kg, b.w.) | | |
| IV | Cyclophosphamide (250 mg/kg, b.w.) + | 4.788 ± 0.11***,## | 5.813 ± 0.12* |
| | 3,8-(OH)₂-DBP (10 mg/kg, b.w.) | | |
| V | Cyclophosphamide (250 mg/kg, b.w.) + | 4.688 ± 0.2***,## | 3.250 ± 0.18**,## |
| | 3-OH-DBP + 3,8-(OH)₂-DBP (12 mg/kg, b.w.) | | |
| VI | Cyclophosphamide (250 mg/kg, b.w.) + | 2.625 ± 0.16***,### | 2.875 ± 0.16***,### |
| | 3-OH-DBP (10 mg/kg, b.w.) | | |
| VII | Cyclophosphamide (250 mg/kg, b.w.) + | 5.625 ± 0.16 | 3.750 ± 0.18*,## |
| | 3,8-(OH)₂-DBP (50 mg/kg, b.w.) | | |
| VIII | Cyclophosphamide (250 mg/kg, b.w.) + | 2.913 ± 0.16***,### | 3.875 ± 0.16*,## |
| | 3-OH-DBP + 3,8-(OH)₂-DBP (60 mg/kg, b.w.) | | |

Water intake (ml) of male 3-OH-DBP, 3,8-(OH)₂-DBP, and combined 3-OH-DBP+3,8-(OH)₂-DBP treated mice are compared with vehicle control and cyclophosphamide treated male mice.

As shown in Table 9, water intake was significantly (p<0.05, p<0.001) decreased in male 3-OH-DBP (for instance 2 mg/kg, b.w.), 3,8-(OH)₂-DBP (50 mg/kg, b.w.), and combined 3-OH-DBP+3,8-(OH)₂-DBP (12 and 60 mg/kg, b.w.) treated mice when compared with vehicle control and cyclophosphamide treated male mice.

TABLE 10
Effect of 3-OH-DBP, 3,8-(OH)₂-DBP, and combined
3-OH-DBP + 3,8-(OH)₂-DBP on water intake of female mice

| | | Water Intake (ml) | |
|---|---|---|---|
| Group | Treatment | Day 0 | Day 7 |
| I | Vehicle Control (0.3% CMC) | 2.203 ± 0.11 | 2.525 ± 0.13 |
| II | Cyclophosphamide (250 mg/kg, b.w.) | 3.620 ± 0.16 | 6.425 ± 0.48 |
| III | Cyclophosphamide (250 mg/kg, b.w.) + | 2.338 ± 0.08 | 2.805 ± 0.12### |
| | 3-OH-DBP (2 mg/kg, b.w.) | | |
| IV | Cyclophosphamide (250 mg/kg, b.w.) + | 2.613 ± 0.10 | 1.750 ± 0.09***,### |
| | 3,8-(OH)₂-DBP (10 mg/kg, b.w) | | |
| V | Cyclophosphamide (250 mg/kg, b.w.) + | 2.700 ± 0.07 | 1.788 ± 0.11***,### |
| | 3-OH-DBP + 3,8-(OH)₂-DBP (12 mg/kg, b.w.) | | |
| VI | Cyclophosphamide (250 mg/kg, b.w.) + | 5.138 ± 0.08*** | 4.525 ± 0.19***,## |
| | 3-OH-DBP (10 mg/kg, b.w) | | |
| VII | Cyclophosphamide (250 mg/kg, b.w.) + | 2.338 ± 0.06 | 1.275 ± 0.11***,### |
| | 3,8-(OH)₂-DBP (50 mg/kg, b.w) | | |
| VIII | Cyclophosphamide (250 mg/kg, b.w.) + | 2.538 ± 0.11 | 1.6 ± 0.14***,### |
| | 3-OH-DBP + 3,8-(OH)₂-DBP (60 mg/kg, b.w.) | | |

Water intake (ml) of female 3-OH-DBP, 3,8-(OH)₂-DBP, and combined 3-OH-DBP+3,8-(OH)₂-DBP treated mice are compared with vehicle control and cyclophosphamide treated female mice.

As shown in Table 10, water intake was significantly (p<0.001) decreased in female 3-OH-DBP (10 mg/kg, b.w.), 3,8-(OH)₂-DBP (10 and 50 mg/kg, b.w.), and combined 3-OH-DBP+3,8-(OH)₂-DBP (12 and 60 mg/kg, b.w.) treated mice when compared with vehicle control female mice. Water intake was significantly (p<0.001) decreased in female 3-OH-DBP (2 and 10 mg/kg, b.w), 3,8-(OH)₂-DBP (10 and 50 mg/kg, b.w), and 3-OH-DBP+3,8-(OH)₂-DBP (12 and 60 mg/kg, b.w.) treated mice when compared with cyclophosphamide treated female mice.

TABLE 11
Effect of 3-OH-DBP, 3,8-(OH)₂-DBP, and combined 3-OH-DBP +
3,8-(OH)₂-DBP on peritoneal macrophage viability in male mice

| | | % Viability |
|---|---|---|
| Group | Treatment | Day 7 |
| I | Vehicle Control (0.3% CMC) | 91.45 ± 0.67 |
| II | Cyclophosphamide (250 mg/kg, b.w.) | 55.72 ± 2.18* |
| III | Cyclophosphamide (250 mg/kg, b.w.) + | 59.90 ± 0.51* |
| | 3-OH-DBP (2 mg/kg, b.w.) | |
| IV | Cyclophosphamide (250 mg/kg, b.w.) + | 60.34 ± 1.84 |
| | 3,8-(OH)₂-DBP (10 mg/kg, b.w) | |
| V | Cyclophosphamide (250 mg/kg, b.w.) + | 62.69 ± 2.21 |
| | 3-OH-DBP + 3,8-(OH)₂-DBP | |
| | (12 mg/kg, b.w.) | |
| VI | Cyclophosphamide (250 mg/kg, b.w.) + | 69.64 ± 2.77# |
| | 3-OH-DBP (10 mg/kg, b.w) | |
| VII | Cyclophosphamide (250 mg/kg, b.w.) + | 70.91 ± 1.24# |
| | 3,8-(OH)₂-DBP (50 mg/kg, b.w) | |
| VIII | Cyclophosphamide (250 mg/kg, b.w.) + | 95.82 ± 0.72*,## |
| | 3-OH-DBP + 3,8-(OH)₂-DBP | |
| | (60 mg/kg, b.w.) | |

Peritoneal macrophage viability (%) of male 3-OH-DBP, 3,8-(OH)₂-DBP, and combined 3-OH-DBP+3,8-(OH)₂-DBP treated mice was compared with vehicle control and cyclophosphamide treated male mice.

As shown in Table 11, peritoneal macrophage viability was significantly (p<0.05) decreased in male cyclophosphamide, 3-OH-DBP (2 mg/kg, b.w.), and combined 3,8-(OH)₂-DBP (10 mg/kg, b.w.) treated mice when compared with vehicle control male mice. Peritoneal macrophage viability was significantly (p<0.05, p<0.01) increased in male 3-OH-DBP (10 mg/kg b.w.; Group VI), 3,8-(OH)₂-DBP (50 mg/kg, b.w.; Group VII), and combined 3-OH-DBP+3,8-(OH)₂-DBP (60 mg/kg, b.w.; Group VIII; 10 mg/kg 3-OH-DBP+50 mg/kg 3,8-(OH)₂-DBP) treated mice when compared with cyclophosphamide treated male mice. DBPs at higher dose levels (Groups VI and VII) showed significant increases in viability over cyclophosphamide alone, and high dose DBPs in combination (Group VIII) showed synergistic activity, restoring peritoneal macrophage viability to levels higher than the untreated vehicle control group.

TABLE 12
Effect of 3-OH-DBP, 3,8-(OH)₂-DBP, and combined 3-OH-DBP +
3,8-(OH)₂- DBP on peritoneal macrophage viability in female mice

| | | % Viability |
|---|---|---|
| Group | Treatment | Day 7 |
| I | Vehicle Control (0.3% CMC) | 90.34 ± 1.51 |
| II | Cyclophosphamide (250 mg/kg, b.w.) | 58.19 ± 0.60** |
| III | Cyclophosphamide (250 mg/kg, b.w.) + | 62.53 ± 2.14** |
| | 3-OH-DBP (2 mg/kg, b.w.) | |
| IV | Cyclophosphamide (250 mg/kg, b.w.) + | 65.78 ± 2.38** |
| | 3,8-(OH)₂-DBP (10 mg/kg, b.w) | |
| V | Cyclophosphamide (250 mg/kg, b.w.) + | 69.17 ± 0.99** |
| | 3-OH-DBP + 3,8-(OH)₂-DBP | |
| | (12 mg/kg, b.w.) | |
| VI | Cyclophosphamide (250 mg/kg, b.w.) + | 72.56 ± 2.01^{#} |
| | 3-OH-DBP (10 mg/kg, b.w) | |
| VII | Cyclophosphamide (250 mg/kg, b.w.) + | 75.27 ± 0.53^{#} |
| | 3,8-(OH)₂-DBP (50 mg/kg, b.w) | |
| VIII | Cyclophosphamide (250 mg/kg, b.w.) + | 94.77 ± 0.83*,^{##} |
| | 3-OH-DBP + 3,8-(OH)₂-DBP | |
| | (60 mg/kg, b.w.) | |

Peritoneal macrophage viability of female 3-OH-DBP, 3,8-(OH)₂-DBP, and combined 3-OH-DBP+3,8-(OH)₂-DBP treated mice was compared with vehicle control and cyclophosphamide treated female mice.

As shown in Table 12, peritoneal macrophage viability was significantly (p<0.05) decreased in female cyclophosphamide treated mice when compared with vehicle control female mice. Peritoneal macrophage viability was significantly (p<0.05, p<0.01) increased in female 3-OH-DBP (2 and 10 mg/kg, b.w.), 3,8-(OH)₂-DBP (10 and 50 mg/kg, b.w.), and combined 3-OH-DBP+3,8-(OH)₂-DBP (12 and 60 mg/kg, b.w.) treated mice when compared with cyclophosphamide treated female mice. DBPs at higher dose levels (Groups VI and VII) showed significant increases in viability over cyclophosphamide alone, and high dose DBPs in combination (10 mg 3-OH-DBP/kg 50 mg 3,8-(OH)₂-DBP/kg: Group VIII) showed synergistic activity, restoring peritoneal macrophage viability to macrophage viability levels significantly higher than the untreated vehicle control group.

TABLE 13
Effect of 3-OH-DBP, 3,8-(OH)₂-DBP, and combined
3-OH-DBP + 3,8-(OH)₂- DBP on NBT reduction
of peritoneal macrophages in male mice

| | | O.D (515 nm) |
|---|---|---|
| Group | Treatment | Day 7 |
| I | Vehicle Control (0.3% CMC) | 0.502 ± 0.04184 |
| II | Cyclophosphamide (250 mg/kg, b.w.) | 0.209 ± 0.02419*** |
| III | Cyclophosphamide (250 mg/kg, b.w.) + | 0.257 ± 0.02338*** |
| | 3-OH-DBP (2 mg/kg, b.w.) | |
| IV | Cyclophosphamide (250 mg/k2, b.w.) + | 0.294 ± 0.04499*** |
| | 3,8-(OH)₂-DBP (10 mg/kg, b.w) | |
| V | Cyclophosphamide (250 mg/kg, b.w.) + | 0.380 ± 0.07001**,# |
| | 3-OH-DBP + 3,8-(OH)₂-DBP | |
| | (12 mg/kg, b.w.) | |
| VI | Cyclophosphamide (250 mg/kg, b.w.) + | 0.232 ± 0.04097*** |
| | 3-OH-DBP (10 mg/kg, b.w) | |
| VII | Cyclophosphamide (250 mg/kg, b.w.) + | 0.476 ± 0.07172## |
| | 3,8-(OH)₂-DBP (50 mg/kg, b.w) | |
| VIII | Cyclophosphamide (250 mg/kg, b.w.) + | 0.519 ± 0.01896### |
| | 3-OH-DBP + 3,8-(OH)₂-DBP | |
| | (60 mg/kg, b.w.) | |

NBT reduction of male 3-OH-DBP, 3,8-(OH)₂-DBP, 3-OH-DBP+3,8-(OH)₂-DBP treated mice are compared with vehicle control and cyclophosphamide treated male mice.

As shown in Table 13, NBT reduction was significantly (p<0.01, p<0.001) decreased in male cyclophosphamide, 3-OH-DBP (2 and 10 mg/kg, b.w), 3,8-(OH)₂-DBP (10 mg/kg, b.w) and combined 3-OH-DBP+3,8-(OH)₂-DBP (12 mg/kg, b.w) treated mice when compared with vehicle control male mice. NBT reduction was significantly (p<0.05, p<0.01, p<0.001) reversed, and NBT reduction capacity increased, in male 3,8-(OH)₂-DBP (50 mg/kg, b.w), and combined 3-OH-DBP+3,8-(OH)₂-DBP (12 and 60 mg/kg, b.w) treated mice when compared with cyclophosphamide treated male mice. Synergistic activity is seen in particular where combined DBPs are present in higher doses (Group VIII), indicating not only the restoration of peritoneal macrophage activity but also a further increase in activity to levels significantly higher than the untreated vehicle control group (Group I).

TABLE 14
Effect of 3-OH-DBP, 3,8-(OH)₂-DBP, and combined
3-OH-DBP + 3,8-(OH)₂-DBP on NBT reduction
of peritoneal macrophages in female mice

| | | O.D. (515 nm) |
|---|---|---|
| Group | Treatment | Day 7 |
| I | Vehicle Control (0.3% CMC) | 0.359 ± 0.06 |
| II | Cyclophosphamide (250 mg/kg, b.w.) | 0.119 ± 0.005*** |
| III | Cyclophosphamide (250 mg/kg, b.w.) + | 0.142 ± 0.005*** |
| | 3-OH-DBP (2 mg/kg, b.w.) | |
| IV | Cyclophosphamide (250 mg/kg, b.w.) + | 0.159 ± 0.02*** |
| | 3,8-(OH)₂-DBP (10 mg/kg, b.w) | |
| V | Cyclophosphamide (250 mg/kg, b.w.) + | 0.162 ± 0.01 ***,# |
| | 3-OH-DBP + 3,8-(OH)₂-DBP | |
| | (12 mg/kg, b.w.) | |
| VI | Cyclophosphamide (250 mg/kg, b.w.) + | 0.207 ± 0.02**,## |
| | 3-OH-DBP (10 mg/kg, b.w) | |
| VII | Cyclophosphamide (250 mg/kg, b.w.) + | 0.294 ± 0.008*,### |
| | 3,8-(OH)₂-DBP (50 mg/kg, b.w) | |
| VIII | Cyclophosphamide (250 mg/kg, b.w.) + | 0.378 ± 0.007### |
| | 3-OH-DBP + 3,8-(OH)₂-DBP | |
| | (60 mg/kg, b.w.) | |

NBT reduction of female 3-OH-DBP, 3,8-(OH)₂-DBP, and combined 3-OH-DBP+3,8-(OH)₂-DBP treated mice were compared with vehicle control and cyclophosphamide treated female mice.

As shown in Table 14, NBT reduction was significantly (p<0.05, p<0.01, p<0.001) decreased in female cyclophosphamide, 3-OH-DBP (2 and 10 mg/kg, b.w), 3,8-(OH)₂-DBP (10 and 50 mg/kg, b.w) and 3-OH-DBP+3,8-(OH)₂-DBP (12 mg/kg, b.w) treated mice when compared with vehicle control female mice. NBT reduction was significantly (p<0.05, p<0.01, p<0.001) increased in female 3-OH-DBP (10 mg/kg, b.w), 3,8-(OH)₂-DBP (50 mg/kg, b.w), 3-OH-DBP+3,8-(OH)₂-DBP (12 and 60 mg/kg, b.w), treated mice when compared with cyclophosphamide treated female mice. Synergistic activity is seen in particular where combined DBPs are present in higher doses (Group VIII), indicating not only the restoration of peritoneal macrophage activity but also a further increase in activity to levels significantly higher than the untreated vehicle control group (Group I).

TABLE 15
Effect of 3-OH-DBP, 3,8-(OH)₂-DBP, and combined 3-OH-
DBP + 3,8-(OH)₂-DBP on splenocyte viability in male mice

| | | % Viability |
|---|---|---|
| Group | Treatment | Day7 |
| I | Vehicle Control (0.3% CMC) | 92.45 ± 0.67 |
| II | Cyclophosphamide (250 mg/kg, b.w.) | 70.22 ± 0.98*** |
| III | Cyclophosphamide (250 mg/kg, b.w.) + | 69.34 ± 1.84*** |
| | 3-OH-DBP (2 mg/kg, b.w.) | |
| IV | Cyclophosphamide (250 mg/kg, b.w.) + | 68.69 ± 2.21*** |
| | 3,8-(OH)₂-DBP (10 mg/kg, b.w) | |
| V | Cyclophosphamide (250 mg/kg, b.w.) + | 71.90 ± 0.51*** |
| | 3-OH-DBP + 3,8-(OH)₂-DBP | |
| | (12 mg/kg, b.w.) | |
| VI | Cyclophosphamide (250 mg/kg, b.w.) + | 80.9 ± 1.24## |
| | 3-OH-DBP(10 mg/kg, b.w) | |
| VII | Cyclophosphamide (250 mg/kg, b.w.) + | 90.14 ± 0.36## |
| | 3,8-(OH)₂-DBP (50 mg/kg, b.w) | |
| VIII | Cyclophosphamide (250 mg/kg, b.w.) + | 95.82 ± 0.72### |
| | 3-OH-DBP + 3,8-(OH)₂-DBP | |
| | (60 mg/kg, b.w.) | |

Splenocyte viability of male 3-OH-DBP, 3,8-(OH)₂-DBP, and combined 3-OH-DBP+3,8-(OH)₂-DBP treated mice was compared with vehicle control and cyclophosphamide treated male mice.

As shown in Table 15, splenocyte viability was significantly (p<0.001) decreased in male cyclophosphamide treated mice when compared with vehicle control male mice, and similarly decreased in groups treated with low dose DBPs (2 mg/kg 3-OH-DBP, 10 mg/kg 3,8-(OH)₂-DBP, and 12 mg/kg combined 3-OH-DBP+3,8-(OH)₂-DBP; Groups III-V). Splenocyte viability in mice treated with cyclophosphamide and higher doses of DBPs was significantly higher in male 3-OH-DBP (10 mg/kg b.w.), 3,8-(OH)₂-DBP (50 mg/kg, b.w), and combined 3-OH-DBP+3,8-(OH)₂-DBP (60 mg/kg b.w) treated mice when compared with cyclophosphamide treated male mice. Splenocyte viability was restored with the 50 mg/kg 3,8-(OH)₂-DBP treatment of Group VII, and with the 60 mg/kg DBP combination of Group VIII.

TABLE 16
Effect of 3-OH-DBP, 3,8-(OH)₂-DBP, and combined 3-OH-
DBP + 3,8-(OH)₂-DBP on splenocyte viability in female mice

| | | % Viability |
|---|---|---|
| Group | Treatment | Day 7 |
| I | Vehicle Control (0.3% CMC) | 91.48 ± 1.74 |
| II | Cyclophosphamide (250 mg/kg, b.w.) | 79.70 ± 1.58*** |
| III | Cyclophosphamide (250 mg/kg, b.w.) + | 88.40 ± 1.61# |
| | 3-OH-DBP (2 mg/kg, b.w.) | |
| IV | Cyclophosphamide (250 mg/kg, b.w.) + | 89.50 ± 2.30# |
| | 3,8-(OH)₂-DBP (10 mg/kg, b.w) | |
| V | Cyclophosphamide (250 mg/kg, b.w.) + | 88.78 ± 1.59 |
| | 3-OH-DBP + 3,8-(OH)₂-DBP | |
| | (12 mg/kg, b.w.) | |
| VI | Cyclophosphamide (250 mg/kg, b.w.) + | 90.77 ± 1.77## |
| | 3-OH-DBP (10 mg/kg, b.w) | |
| VII | Cyclophosphamide (250 mg/kg, b.w.) + | 90.87 ± 1.98## |
| | 3,8-(OH)₂-DBP (50 mg/kg, b.w) | |
| VIII | Cyclophosphamide (250 mg/kg, b.w.) + | 96.29 ± 1.15### |
| | 3-OH-DBP + 3,8-(OH)₂-DBP | |
| | (60 mg/kg, b.w.) | |

Splenocyte viability of female 3-OH-DBP, 3,8-(OH)₂-DBP, and combined 3-OH-DBP+3,8-(OH)₂-DBP treated mice were compared with vehicle control and cyclophosphamide treated female mice.

As shown in Table 16, splenocyte viability was significantly (p<0.001) decreased in female cyclophosphamide treated mice when compared with vehicle control female mice. Splenocyte viability was significantly (p<0.05, p<0.01, p<0.001) increased in female 3-OH-DBP (2 and 10 mg/kg b.w.), 3,8-(OH)₂-DBP (10 and 50 mg/kg, b.w) 3-OH-DBP+3,8-(OH)₂-DBP (12 and 60 mg/kg, b.w) treated mice when compared with cyclophosphamide treated female mice.

### CONCLUSION

From the above findings it can be concluded that 3-OH-DBP and 3,8-(OH)₂-DBP increased macrophage viability and NBT reduction and splenocyte viability. DBP combinations at the higher dose level exhibited synergistic activity.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All method steps described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification the present invention has been described in relation to certain embodiments thereof, and many details have been put forth for the purposes of illustration, it will be apparent to those skilled in the art that the invention is susceptible to additional embodiments and that certain of the details described herein can be varied considerably without departing from the basic principles of the invention.

The present invention may be embodied in other specific forms without departing from the spirit or essential attributes thereof, and, accordingly, reference should be made to the appended claims, rather than to the foregoing specification, as indicating the scope of the invention.

1. A method of improving a subject's immune system comprising the steps of:
(a) providing a composition comprising at least one of 3-OH-DBP, 3-OH-DBP glycoside, 3,8-(OH)₂-DBP, and 3,8-(OH)₂-DBP glycoside; and
(b) administering the composition to the subject in an amount effective to deliver the at least one of 3-OH-DBP, 3-OH-DBP glycoside, 3,8-(OH)₂-DBP, and 3,8-(OH)₂-DBP glycoside to the subject's cells, tissues, and/or bloodstream and to act at said cells, tissues, and/or bloodstream to improve an immune system parameter.

2. The method of claim 1, wherein the effective amount of 3-OH-DBP, 3-OH-DBP glycoside, 3,8-(OH)₂-DBP, and/or 3,8-(OH)₂-DBP glycoside is at least 1 daily dose of about 0.5 mg to about 75 mg per kilogram body weight of the subject.

3. The method of claim 2, wherein the composition comprises at least one of 3-OH-DBP and 3-OH-DBP glycoside, and also at least one of 3,8-(OH)₂-DBP and 3,8-(OH)₂-DBP glycoside.

4. The method of claim 3, wherein the composition comprises a ratio of (3-OH-DBP and/or 3-OH-DBP glycoside) to (3,8-(OH)₂-DBP and/or 3,8-(OH)₂-DBP glycoside) of about 1:3 to about 1:7 (w/w).

5. The method of claim 4, wherein the ratio is 1:5.

6. The method of claim 4, wherein the composition comprises 3-OH-DBP and 3,8-(OH)₂-DBP in said ratio, or 3-OH-DBP glycoside and 3,8-(OH)₂-DBP glycoside in said ratio.

7. The method of claim 1, wherein the composition is administered daily for at least 7 days.

8. The method of claim 1, wherein said immune system is improved by increasing an immune system parameter.

9. The method of claim 8, wherein said increased immune system parameter is increased viability of macrophages, increased activity of macrophages, and/or increased viability of splenocytes.

10. The method of claim 8, wherein said immune system is immunosuppressed, and said immunosuppressed immune system is improved by increasing the viability of macrophages, activity of macrophages, and/or viability of splenocytes.

11. The method of claim 10, wherein said immune system is immunosuppressed by cyclophosphamide.

12. The method of claim 1, wherein said subject is human.

13. A method of providing immuno-protection to a subject, comprising the steps of:
(a) providing a composition comprising at least one of 3-OH-DBP, 3-OH-DBP glycoside, 3,8-(OH)₂-DBP, and 3,8-(OH)₂-DBP glycoside; and
(b) administering the composition to the subject in an amount effective to deliver the at least one of 3-OH-DBP, 3-OH-DBP glycoside, 3,8-(OH)₂-DBP, and 3,8-(OH)₂-DBP glycoside to the subject's cells, tissues, and/or bloodstream and to act at said cells, tissues, and/or bloodstream to maintain the subject's immune response at normal healthy levels.

14. The method of claim 13, wherein the effective amount of 3-OH-DBP, 3-OH-DBP glycoside, 3,8-(OH)₂-DBP, and/or 3,8-(OH)₂-DBP glycoside is at least 1 daily dose of about 0.5 mg to about 75 mg per kilogram body weight of the subject.

15. The method of claim 14, wherein the composition comprises a ratio of (3-OH-DBP and/or 3-OH-DBP glycoside) to (3,8-(OH)₂-DBP and/or 3,8-(OH)₂-DBP glycoside) of about 1:3 to about 1:7 (w/w).

16. The method of claim 15, wherein the ratio is 1:5.

17. A compound having the chemical structure of Formula (I): 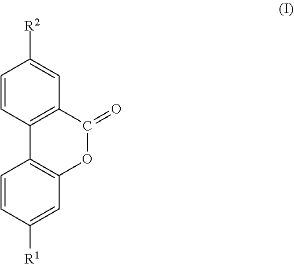
wherein R¹═—O-sugar and R²═H, or wherein R¹═—O-sugar and R²═—O-sugar.

18. The compound of claim 17, wherein R¹═—O-sugar and R²═H, and said sugar is a monosaccharide or a disaccharide.

19. The compound of claim 18, wherein said sugar is a monosaccharide, and said monosaccharide is glucose, xylose, rhamnose, arabinose, or galactose.

20. The compound of claim 18, wherein said sugar is a disaccharide, and said disaccharide is a combination of glucose, xylose, rhamnose, arabinose, and/or galactose.

21. The compound of claim 17, wherein R¹═—O-sugar and R²═—O-sugar and each sugar is independently a monosaccharide or a disaccharide.

22. The compound of claim 21, wherein if present said monosaccharide is glucose, xylose, rhamnose, arabinose, or galactose, and if present said disaccharide is a combination of glucose, xylose, rhamnose, arabinose, and/or galactose.

23. The compound of claim 17, wherein R¹═—O-glucose and R²═H, having the chemical structure of Formula (II), 3-O-D-Glucopyranosyl-Dibenzo-alpha-pyrone:

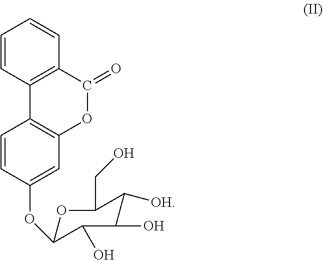 [SMILES: O=c1oc2cc(OC3OC(CO)C(O)C(O)C3O)ccc2c2ccccc12]

24. The compound of claim 17, wherein R¹═—O-glucose and R²═—O-glucose, having the chemical structure of Formula (III), 3,8-di-(O-D-Glucopyranosyl)-Dibenzo-alpha-pyrone:

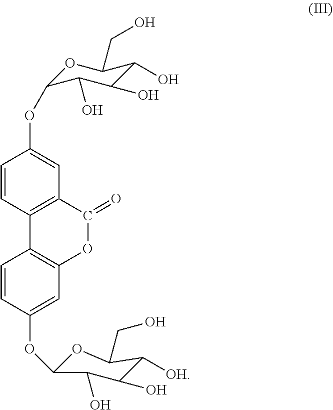 [SMILES: O=c1oc2cc(OC3OC(CO)C(O)C(O)C3O)ccc2c2ccc(OC3OC(CO)C(O)C(O)C3O)cc12]

25. A composition comprising a compound of claim 17.

26. The composition of claim 25, further comprising microcrystalline cellulose, croscarmellose sodium, magnesium stearate, and/or silicon dioxide.

27. A process for preparing a DBP glycoside comprising the steps of:
a. acetylating a sugar,
b. brominating the acetylated sugar, and
c. adding the acetylated brominated sugar and a DBP to prepare the DBP glycoside.

28. The process of claim 27, wherein said sugar is glucose, xylose, rhamnose, arabinose, galactose, or a disaccharide containing glucose, xylose, rhamnose, arabinose, and/or galactose.

29. The process of claim 27, wherein said DBP glycoside is a compound of claim 17.

30. The process of claim 27, wherein said sugar is glucose, said acetylated brominated sugar is acetyl glucopyranosyl bromide, said DBP is 3-OH-DBP, and said DBP glycoside is the 3-OH-DBP glycoside compound of Formula II.

31. The process of claim 27, wherein said sugar is glucose, said acetylated brominated sugar is acetyl glucopyranosyl bromide, said DBP is 3,8-(OH)₂-DBP, and said DBP glycoside is the 3,8-(OH)₂-DBP glycoside compound of Formula III.

32. The process of claim 27, further comprising the step of purifying the DBP glycoside.
